SEQUENCE LISTING (33 sequences)

SEQ ID NO: 1 - DNA, 23 bp, Artificial Sequence
  Primer
gaatttgtaatttcgtgcgtttc

SEQ ID NO: 2 - DNA, 22 bp, Artificial Sequence
  Primer
aaaaaaaactctccgtacaccg

SEQ ID NO: 3 - DNA, 25 bp, Artificial Sequence
  Primer
tgaatttgtaattttgtgtgttttg

SEQ ID NO: 4 - DNA, 24 bp, Artificial Sequence
  Primer
aaaaaaaactctccatacaccacc

SEQ ID NO: 5 - DNA, 24 bp, Artificial Sequence
  Primer
ttaattttagagtggagaacgtgc

SEQ ID NO: 6 - DNA, 20 bp, Artificial Sequence
  Primer
aaataacaatacgacccgcc

SEQ ID NO: 7 - DNA, 22 bp, Artificial Sequence
  Primer
ttttagagtggagaatgtgtgt

SEQ ID NO: 8 - DNA, 25 bp, Artificial Sequence
  Primer
aaacaaaataacaatacaacccacc

SEQ ID NO: 9 - DNA, 25 bp, Artificial Sequence
  Primer
gaagagagttggtgttaagagttag

SEQ ID NO: 10 - DNA, 19 bp, Artificial Sequence
  Primer
ccaccaaaaaaaactctcc

SEQ ID NO: 11 - DNA, 18 bp, Artificial Sequence
  Primer
tgtgtgtttgaggatttg

SEQ ID NO: 12 - DNA, 1848 bp, Homo sapiens
cacgctgcgcccggcgccgcagggtgggcggcgaggcactcacgagggggacgctgagggcttccgcgcgggccacccgggtcaagcgcgctccgccgggaaacttctgcgggcgccgggctgaagctccgggcagggctgggaaggaaaggaaataccaaaatatttgcagactggatccaaatcaggagcccagatgaacttaaagaagcgagcatgggattcttagtttttcaagatccgtacacacgaagcctttaatcagcatcaactccagtgtccgttttctctggttttgtgaagactgcacaaaactctcatgatggagaaccaaaggacttagttacctgtttcagtgtcatctaaagtcaactgaaaagtgaagcaggcagtaatacagccatggaaagaaattcaagtttatggaagaacctaatagatgaacacccagtctgcacaacctggaagcaagaggccgaaggagccatttatcatcttgccagtattttatttgtagtaggtttcatgggtggcagtggattcttcgggctcctttatgtcttcagtttgctggggttgggttttctctgttctgctgtctgggcttgggtagatgtctgtgcagctgacatattttcctggaattttgtactgtttgtcatctgcttcatgcaatttgttcatattgcatatcaagttcgcagcataacctttgcccgagaattccaagtgttgtacagctcccttttccagcccctggggatctctttgcctgtcttcagaacgattgctttgagctctgaagtggttactttggaaaaggaacactgttatgccatgcaggggaaaacttccattgataaactctccttgcttgtttcaggaaggatcagagtgacagttgatggcgaatttctgcattacattttcccccttcagttcctggattctcctgagtgggattcactgagacccacagaggaaggcatttttcaggtaaccctcactgcagaaactgattgtcgatatgtgtcttggaggagaaagaaattatatctgctctttgctcagcatcgctacatctcccgccttttttcagtgctaattggcagtgacattgcagataaactctatgccttgaatgacagggtatatataggaaaaagatatcactatgatattcggctacccaacttctatcaaatgtcaactccagaaatacgcagatcacccctgacacaacattttcagaattccagacgatactgtgataaatgacatcaaagtctgaaatttataagtataaaaaaagactctctcttcatcattccccagtgaaatagcaaaatacaaaaaaagagctccctaatgtttttataaatcaaattcagaagcgagatgccattgccaactgttttattcctttcaacaactgcattgtgaataaactttacaaatttttcttgtatttctcattgttataattggggagggggtggataactatgggcagtttgcccttttctgcatcaaaactgggagaatgaaattccactttctcaattcttttctcacatttactcaaatgcattgtcttgccctatagactcaggagttgcttctcaagaaagagccagcaagtattctcagcctgagggtgggttgctactgtccacataggcatttccggaattcacttttttgctacaacctccagtgaaagcaattatttattttaaatgtgcagttacttgatgcgactaaaaagtagaataaatgcaagagataataatatgtgaatcttgaagcctattttattgctacaaataaaataacatttaaaaagaa

SEQ ID NO: 13 - DNA, 2698 bp, Homo sapiens
atacgcggtcctgcgccctcgcctcagacctctcgggcgagcgcggcgcagcgcagattaaaaatcaagaaatataaaccagatgtagcagtttcttgacatggagaaccaagcccataatacgatgggaacttctccttgtgaggctgagcttcaggaattaatggaacaaattgacatcatggtaagcaacaagaaaatggattgggaaagaaagatgcgggctttggagacacgattagatcttcgggatcaagaattggcaaatgcacaaacttgtttggatcagaaaggtcaagaggtagggttacttcgacagaaattggacagtctggaaaaatgtaatttagcaatgactcagaattatgaaggacaactacaaagcctaaaggctcaattttccaaactaacaaataactttgaaaaactgagattacatcagatgaaacaaaacaaagttccacgaaaagaattaccacaccttaaagaagaaataccctttgaactgagcaatttgaaccagaaattagaggaatttagagcaaagtcaagagaatgggacaagcaagagatattatatcagactcatctgatttctttagatgctcaacaaaaattattatctgagaagtgtaatcagtttcagaaacaggcacaaagttaccaaactcaactaaatggtaaaaaacagtgcttagaagacagcagctctgaaattcctcgtttgatatgtgacccagatcccaattgtgaaatcaatgaaagagatgagttcattattgaaaaactgaaatcagctgtaaatgagatagcactaagcaggaataaattacaagatgaaaatcagaagctcttgcaagaactgaaaatgtaccaaagacagtgccaggccatggaagcaggtctctcagaggtaaaaagtgagttacagtcacgtgatgatctcttgagaattatagaaatggaacgattgcaattacacagagaattattaaaaataggagagtgccaaaatgctcaaggaaataaaacaagacttgaatcatcttatttgccttctattaaagaaccagaaaggaaaataaaagagctgttttcagtgatgcaagatcaaccaaatcatgaaaaagaattgaacaagataagaagccaactccaacaggtggaagagtaccataactctgagcaggaaagaatgaggaatgaaatctctgacctaacagaagagcttcatcagaaggagatcactatagcaactgtcacaaagaaagctgcccttctggaaaaacagttaaaaatggaattagaaataaaagaaaaaatgttagcaaaacaaaaggtctcagatatgaaatataaagctgtcagaactgaaaacacacatctaaaaggaatgatgggagatttagaccccggagaatacatgagtatggacttcactaacagggaacagtcaaggcatacatctattaataaactgcaatatgagaatgaaaggctccgaaatgatcttgcaaaacttcatgtcaatggaaaatcaacctggactaatcaaaacacctatgaagaaacaggaagatatgcctatcaaagccaaataaaagtggaacaaaatgaagagagacttagtcatgactgtgagccaaacagaagtacaatgcctcccttgccaccttcgacatttcaagccaaagaaatgacaagtcctttggttagtgatgatgatgtattcccactgtctcccccagatatgtccttcccagcatctttggctgcacagcatttccttctggaagaagagaaacgagcaaaagaacttgaaaaacttctaaatacacatattgatgaactgcaaagacacacagaatttactcttaataaatactccaagctaaaacaaaatagacacatatgagcttttaaacttttttatttgcttcccccccccacccccgccaagaaaaaaagctctggcaaaatatttacaaagctgattaatgaaaccaagaaattctgttctgtttccttgagtaaataatttccgtaaggcagctagaaaatagtaagtattttgttctataaagctgttcacatttctgcattaacatgctaaattgtcctgctgtagagttactataaaataaacatgactattccaaaagagatttttttccagtctaaggaatattttggaatcaaatatgcagttttttttccccacttgaatcatgtatactgaaaacccatttcctcagcaactacacaaaataagagaaagggaagtgctatattccagatacaaatgactcaaaggcagctcagttgtactggttttgaaaactcaaggattttataaataaaaatatttaagtatttccagttatgtaaacactctaaaattctataattttttggaaaaaaaaagctatagctttattgtttttacattcacctttataatgtctgtctgtttcaacacaatgaaggtatatggaatgtgcaatttacaaacctaaaatatatgtgccaaaaattaacgcttttttgttagtactaattacttcatggacacaaacaagataaaggaaatataaagcattactgttgctacattttcctttgtaatcacttctgtttttaatacaataaaggaatgtaaaattcaaaaaaaaaaaaaaaaaaaaaaaaaaaaaaaaaa

SEQ ID NO: 14 - DNA, 1718 bp, Homo sapiens
gactgagccgactgcatctctgggcaagcggcgctttgcctctctcctgtctttgtctgctcttcctgcaaggctacggcgcctggagcagggtctgcagcggccgccgcagcaacgcgagccaagtcgtgccccgcacggccgcccggggccgcaccctcgctcggtggcggcgcccgaagacgccagccgcgccacgcactgcctgcgtcccgcgccccagccgccgcgcaccaacgccgccgccttcgccgggagccaagcccgccgggccggcccggtcccaggtgggcagctcggcttgcaactggttggaggtggcggccgctgcagccctggcagcgggcacacccctaagcatacgcacccaacgcctcctccctgagccacgaggatggagcagccaccccggcccgggactggcgcaaggtgcccaagcaaggaaagaaataatgaagagacacatgtgttagctgcagccttttgaaacacgcaagaaggaaatcaatagtgtggacagggctggaacctttaccacgcttgttggagtagatgaggaatgggctcgtgattatgctgacattccagcatgaatctggtagacctgtggttaacccgttccctctccatgtgtctcctcctacaaagttttgttcttatgatactgtgctttcattctgccagtatgtgtcccaagggctgtctttgttcttcctctgggggtttaaatgtcacctgtagcaatgcaaatctcaaggaaatacctagagatcttcctcctgaaacagtcttactgtatctggactccaatcagatcacatctattcccaatgaaatttttaaggacctccatcaactgagagttctcaacctgtccaaaaatggcattgagtttatcgatgagcatgccttcaaaggagtagctgaaaccttgcagactctggacttgtccgacaatcggattcaaagtgtgcacaaaaatgccttcaataacctgaaggccagggccagaattgccaacaacccctggcactgcgactgtactctacagcaagttctgaggagcatggcgtccaatcatgagacagcccacaacgtgatctgtaaaacgtccgtgttggatgaacatgctggcagaccattcctcaatgctgccaacgacgctgacctttgtaacctccctaaaaaaactaccgattatgccatgctggtcaccatgtttggctggttcactatggtgatctcatatgtggtatattatgtgaggcaaaatcaggaggatgcccggagacacctcgaatacttgaaatccctgccaagcaggcagaagaaagcagatgaacctgatgatattagcactgtggtatagtgtccaaactgactgtcattgagaaagaaagaaagtagtttgcgattgcagtagaaataagtggtttacttctcccatccattgtaaacatttgaaactttgtatttcagtttcttttgaattatgccactgctgaacttttaacaaacactacaacataaataatttgagtttaggtgatccaccccttaattgtacccccgatggtatatttttgagtaagctactatttgaacattagttagatccatctcactatttaataatgaaatttatttttttaatttaaaagcgaataaaaggttaactttgaaccatggaaaaaaaaaaaaaaaaaaaaaaaaaaaaa

SEQ ID NO: 15 - DNA, 3679 bp, Homo sapiens
ccctcccctcccgatcctcatccccttgccctcccccagcccagggacttttccggaaagtttttattttccgtctgggctctcggagaaagaagctcctggctcagcggctgcaaaactttcctgctgccgcgccgccagcccccgccctccgctgcccggccctgcgccccgccgagcgatgagcgcccctccggtcctgcggccgcccagtccgctgctgcccgtggcggcggcagctgccgcagcggccgccgcactggtcccagggtccgggcccgggcccgcgccgttcttggctcctgtcgcggccccggtcgggggcatctcgttccatctgcagatcggcctgagccgtgagccggtgctgctgctgcaggactcgtccggggactacagcctggcgcacgtccgcgagatggcttgctccattgtcgaccagaagttccctgaatgtggtttctacggaatgtatgataagatcctgctttttcgccatgaccctacctctgaaaacatccttcagctggtgaaagcggccagtgatatccaggaaggcgatcttattgaagtggtcttgtcagcttccgccacctttgaagactttcagattcgtccccacgctctctttgttcattcatacagagctccagctttctgtgatcactgtggagaaatgctgtgggggctggtacgtcaaggtcttaaatgtgaagggtgtggtctgaattaccataagagatgtgcatttaaaatacccaacaattgcagcggtgtgaggcggagaaggctctcaaacgtttccctcactggggtcagcaccatccgcacatcatctgctgaactctctacaagtgcccctgatgagccccttctgcaaaaatcaccatcagagtcgtttattggtcgagagaagaggtcaaattctcaatcatacattggacgaccaattcaccttgacaagattttgatgtctaaagttaaagtgccgcacacatttgtcatccactcctacacccggcccacagtgtgccagtactgcaagaagcttctgaaggggcttttcaggcagggcttgcagtgcaaagattgcagattcaactgccataaacgttgtgcaccgaaagtaccaaacaactgccttggcgaagtgaccattaatggagatttgcttagccctggggcagagtctgatgtggtcatggaagaagggagtgatgacaatgatagtgaaaggaacagtgggctcatggatgatatggaagaagcaatggtccaagatgcagagatggcaatggcagagtgccagaacgacagtggcgagatgcaagatccagacccagaccacgaggacgccaacagaaccatcagtccatcaacaagcaacaatatcccactcatgagggtagtgcagtctgtcaaacacacgaagaggaaaagcagcacagtcatgaaagaaggatggatggtccactacaccagcaaggacacgctgcggaaacggcactattggagattggatagcaaatgtattaccctctttcagaatgacacaggaagcaggtactacaaggaaattcctttatctgaaattttgtctctggaaccagtaaaaacttcagctttaattcctaatggggccaatcctcattgtttcgaaatcactacggcaaatgtagtgtattatgtgggagaaaatgtggtcaatccttccagcccatcaccaaataacagtgttctcaccagtggcgttggtgcagatgtggccaggatgtgggagatagccatccagcatgcccttatgcccgtcattcccaagggctcctccgtgggtacaggaaccaacttgcacagagatatctctgtgagtatttcagtatcaaattgccagattcaagaaaatgtggacatcagcacagtatatcagatttttcctgatgaagtactgggttctggacagtttggaattgtttatggaggaaaacatcgtaaaacaggaagagatgtagctattaaaatcattgacaaattacgatttccaacaaaacaagaaagccagcttcgtaatgaggttgcaattctacagaaccttcatcaccctggtgttgtaaatttggagtgtatgtttgagacgcctgaaagagtgtttgttgttatggaaaaactccatggagacatgctggaaatgatcttgtcaagtgaaaagggcaggttgccagagcacataacgaagtttttaattactcagatactcgtggctttgcggcaccttcattttaaaaatatcgttcactgtgacctcaaaccagaaaatgtgttgctagcctcagctgatccttttcctcaggtgaaactttgtgattttggttttgcccggatcattggagagaagtctttccggaggtcagtggtgggtacccccgcttacctggctcctgaggtcctaaggaacaagggctacaatcgctctctagacatgtggtctgttggggtcatcatctatgtaagcctaagcggcacattcccatttaatgaagatgaagacatacacgaccaaattcagaatgcagctttcatgtatccaccaaatccctggaaggaaatatctcatgaagccattgatcttatcaacaatttgctgcaagtaaaaatgagaaagcgctacagtgtggataagaccttgagccacccttggctacaggactatcagacctggttagatttgcgagagctggaatgcaaaatcggggagcgctacatcacccatgaaagtgatgacctgaggtgggagaagtatgcaggcgagcaggggctgcagtaccccacacacctgatcaatccaagtgctagccacagtgacactcctgagactgaagaaacagaaatgaaagccctcggtgagcgtgtcagcatcctctgagttccatctcctataatctgtcaaaacactgtggaactaataaatacatacggtcaggtttaacatttgccttgcagaactgccattattttctgtcagatgagaacaaagctgttaaactgttagcactgttgatgtatctgagttgccaagacaaatcaacagaagcatttgtattttgtgtgaccaactgtgttgtattaacaaaagttccctgaaacacgaaacttgttattgtgaatgattcatgttatatttaatgcattaaacctgtctccactgtgcctttgcaaatcagtgtttttcttactggagcttcattttggtaagagacagaatgtatctgtgaagtagttctgtttggtgtgtcccattggtgttgtcattgtaaacaaactcttgaagagtcgattatttccagtgttctatgaacaactccaaaacccatgtgggaaaaaaatgaatgaggagggtagggaataaaatcctaagacacaaatgcatgaacaagttttaatgtatagttttgaatcctttgcctgcctggtgtgcctcagtatatttaaactcaagacaatgcacctagctgtgcaagacctagtgctcttaagcctaaatgccttagaaatgtaaactgccatatataacagatacatttccctctttcttataatactctgttgtactatggaaaatcagctgctcagcaacctttcacctttgtgtatttttcaataataaaaaatattcttgtcaaaa

SEQ ID NO: 16 - DNA, 5160 bp, Homo sapiens
aaaacccggaggagcgggatggcgcgctttgactctggagtgggagtgggagcgagcgcttctgcgactccagttgtgagagccgcaagggcatgggaattgacgccactcaccgacccccagtctcaatctcaacgctgtgaggaaacctcgactttgccaggtccccaagggcagcggggctcggcgagcgaggcacccttctccgtccccatcccaatccaagcgctcctggcactgacgacgccaagagactcgagtgggagttaaagcttccagtgagggcagcaggtgtccaggccgggcctgcgggttcctgttgacgtcttgccctaggcaaaggtcccagttccttctcggagccggctgtcccgcgccactggaaaccgcacctccccgcagcatgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagcagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtataatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaaggaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 17 - DNA, 2173 bp, Homo sapiens
atcctttctatttggagttgactctgctgccctttggagatcccagcgcgtaacgtgagctcgcgaccagcgcgggggaggcgcccacacgcccgtcccgtcttggtgtctggccccaggcggccgcgcctcccctccgaggtggtcccatagcccctgcaccaggcggcccgcgcccgcgacctcaaggcggctgccccgcgccatggcggcccggctgggtgcgctggccgcgtcggggctgtaccggcggcgccagcaccggcagagcccgccgccagccacgggcaatatgtcggacgccttggccaacgccgtgtgccagcgctgccaggcccgcttctcccccgccgagcgcattgtcaacagcaatggggagctgtaccatgagcactgcttcgtgtgtgcccagtgcttccggcccttccccgaggggctcttctatgagtttgaaggccggaagtactgcgaacacgacttccaaatgctgtttgctccgtgctgtggatcctgcggtgagttcatcattggccgcgtcatcaaggccatgaacaacaactggcacccgggctgcttccgctgcgagctgtgtgatgtggagctggctgacctgggctttgtgaagaatgccggcaggcatctctgccggccttgccacaaccgtgagaaggccaagggcctgggcaagtacatctgccagcggtgccacctggtcatcgacgagcagcccctcatgttcaggagcgacgcctaccaccctgaccacttcaactgcacccactgtgggaaggagctgacagccgaggcccgcgagctgaagggtgagctctactgcctgccctgccatgacaagatgggcgtccccatctgcggggcctgccgccggcccatcgagggccgagtggtcaacgcgctgggcaagcagtggcacgtggagcactttgtctgtgccaagtgtgagaagccattcctggggcaccggcactatgagaagaagggcctggcctactgcgagactcactacaaccagctcttcggggacgtctgctacaactgcagccatgtgattgaaggcgatgtggtgtcggccctcaacaaggcctggtgtgtgagctgcttctcctgctccacctgcaacagcaagctcaccctgaagaacaagtttgtggagttcgacatgaagcccgtgtgtaagaggtgctacgagaagttcccgctggagctgaagaagcggctgaagaagctgtcggagctgacctcccgcaaggcccagcccaaggccacagacctcaactctgcctgaaggccctcttgcgcagctgcctctcggcccctccgccttctcccctcctgctgtccatgcttggccccctcgtccccatccacctgtgccctccgcatcttaccctccctttctctttcctcattgccttctcccttcctgttccctcatctctgccttccccatgtctctcctctccttggccgtggcttctgtctgtgaggaggcaggagctggggagtgggagcctatgaccccacgtctgacagccatgtccacctgtgcccacagcttccgcccacagacctccagggacaggagcaaattgcaccacagctccccgcctggcctggccctccccaggcggctcagtggctcatgctgtcctgtgagagcccctgccccagagcggccccactaagcgcatgtggctcctgggctacccacagccagggcagcctgctggagccacagggccagggccatgcagatggaggcctctgggagccacctccaatccctcaccactcactcaaccagtggcacagtgtccttgtgcccacactgagccagcaagtcctgctgtccacacccacaagctacctggagggacaggacccacctccatccttcggaaggccttcctggaatcccaccttggcctccgccctcggttccgccccgcccctctccccccgaccttggggcttgtgtcgagcccttgggtggggccaggaggaggtgatggcgtcagaggaggtgtggtcagaggtgacttgttcccacctccagggaggacgcttcgtcttcggccagcgcagacctggtgtttgtttgttgggctcacgcttgcacaatgaaggcttgttcacacaaccacaaaaaaaaaaa

SEQ ID NO: 18 - DNA, 6093 bp, Homo sapiens
gcctgccagctagccggagccgcgggtgagcgcggcgagcggcgaccctggtgaggagcgcggcgcgggaggcacgttccttagctccgccgcggccgtcctccgcggctcgaggactccgcttccttccctcccctcccctgcgctccggcctggggtctcggcgcggggagcggagggaagggacgaaggaggagtaggtgaaagcggggtgaggggcggaagggtcccggcgcggggtgaggcgagggctgcctcttgttctcccgccgctgccgccgtctcctggtcgggtgccgcggccagaggcgcgcggggctgccgaggcacccgcactatgcaggcagactgccggccgccgcgatggcgagccgggcggtggtgagagccaggcgctgcccgcagtgtccccaagtccgggccgcggccgccgcccccgcctgggccgcgctccccctctcccgctccctccctccctgctccaactcctcctccttctccatgcctctgttcctcctgctcttacttgtcctgctcctgctgctcgaggacgctggagcccagcaaggtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaataggcaaatactgtggtctggggttgcaaatgaaccattcaattgaatcaaaaggcaatgaaatcacattgctgttcatgagtggaatccatgtttctggacgcggatttttggcctcatactctgttatagataaacaagatctaattacttgtttggacactgcatccaattttttggaacctgagttcagtaagtactgcccagctggttgtctgcttccttttgctgagatatctggaacaattcctcatggatatagagattcctcgccattgtgcatggctggtgtgcatgcaggagtagtgtcaaacacgttgggcggccaaatcagtgttgtaattagtaaaggtatcccctattatgaaagttctttggctaacaacgtcacatctgtggtgggacacttatctacaagtctttttacatttaagacaagtggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggcattataaccactggatccaccatggtggagcacaattactatgtgtctgcctacagaatcctgtacagtgatgatgggcagaaatggactgtgtacagagagcctggtgtggagcaagataagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtcgtcctccaaaacttactcaacctccacctcctcggaacagcaatgacctcaaaaacactacagcccctccaaaaatagccaaaggtcgtgccccaaaatttacgcaaccactacaacctcgcagtagcaatgaatttcctgcacagacagaacaaacaactgccagtcctgatatcagaaatactaccgtaactccaaatgtaaccaaagatgtagcgctggctgcagttcttgtccctgtgctggtcatggtcctcactactctcattctcatattagtgtgtgcttggcactggagaaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgctttttacaaagcatcgttttaaagcacatggccttttttttttaattattagtggtagtaatatatagaatgtattacataactgtcactgaagtggttggggaaaatgtggtgactgaggtacaggaaactactaatcttgccatcttgctttaaggtgttatggtggcacagttactgctcgcctgttaaatttcaaatgtcctgtttgatactactgtagaacactatttttaatacagaaaaagctccctataatgcacttcagagaaattaaaaatcacagagtatttattaccaatgctgcaggtacattaatgaactcgagatggctctgtaagcctgactggcaataacgcacggtactgttcttgaaatacctaatggcttgaaattctagtctgtttgtgaaagatgggtactatcatgatttcctcttctattcctatattcttttctggattttttttaataattagtgatataagcattgtttttattgcagccatatccacttatccatcttaagatctgtagctgggattttctgacttgtaatgagcagggggattgctttttcactttgtgacactctttagagctttaatgcttcacagtatatggcctggtctcatccttgcgtgttccacttgaggccctttggtgtcttgccccattcttgtgtttataaaatgtttgagtatttctgatgagtgatgcttgccttagtctcatgaattcagatcccttcatgtcctttaagtatgctcctcaatgtgtaaacaggaacaactttatgatttgaaagctttaaaggagattcttctcccacccccaactttatttgcaatgggatttttcctaggagagttatgaaaagttgaaggcttctaagggaatactgtaaacatgacccacttatatttatcacagtgaaaggcaaaattattcactcagaagtaatataaattacctctttaaaaagtaaccagaatttgtcctttttggttttatacattcacaaacatatacatttttcttgagtctcaaggtattttatatttttagtcagaaaaaataatttttcatttcagttttccataaactgttacacaaaatataaacctaacgtgtatttttcaggactgcgtgatcgtgcactttgtgtggtaagaggtttgagtagtcctatatgtcacctagggaacagacattatagcttactagcaaatgaatattcatgccttgtttttgatacctcctggcagcttccatgtcaccacttgttcatacctgcccagagctagttttagacatggcaaaatagaaatcatctgtaatttattagctaacaatgtaaaaccatcttttaaagccttcagactgtcaagacgacatgagcagctcaccatatgataaaaatacataaatttgacattccctcttccataaacctttgtttgtagatttaatgttgaacagtacttttccataaagttctagtcacttctgttggcctgagccaccagattatgatgttgccagaattcactcaatttgaataaagatgaacagtatttgttttcttgtttccatgaattatatcagtattctaaaacatcgcttcagaaagagaactgtttatttctgcaggcttcctgtccttttgtggtatggttttttggccttattttcactggcttttccttctccaaactttgaggcgtgatttcattcattgaagaatcaatacatattttgtttcaaaatgtttgaaacaaaagacatagatggtagacttttattaaaacatatatggatgtggaaagcacatatattaatgcagtcatcccttttcaggtgggaagagagcaaaccagttgattttttaattcatccttagtacacagagaatatacttttcctcaagtaatatacctgtttgaagctttaagagagatgtttttggtaactatttcattttcccaaagaagtttgctattcttgtgttaattgtgtatacctgattgttttttcctggaggtttttgttgttgttgtttagttttgggtttttttttttttaagaggggcaagtgttttctgaaatgatgcatattttaagactcgattcatattgccactgtgctatccttgaactaccaataatttttataaaatatctagtttttactacttttatataaactttactttccagatgaagagctgagcctgattcaaatggtttttctgctttatacttctttttagttcattggtttttatagtagaggttttctattttttttttttttttttttactacatttatatgtctgatacatatacggctttggagacaatcaagtaacaactgaaaatgtgaaagtaaccatatctgacaaaattcccttgaatttttatcctttgcttgcaacatttaagactcaaagtcactggtatattggattaagttttttcctgttaatgcaattatagaaatacatcggagacacaacaaatgtggccattacaggtttcataaaattacactgacttggctgttacttgatcttaggaaacagcacagtttaagatattgtgaattctgacttatactttattaaatgctgtaaatctaaatagatcctgttggatgtgatgggtctagtccagtttatttaagttcatgtttcactgtttgcactttgcattgaacaatgggtttattcgctgatgtaaacggttcgagtgaagaattaatgcagtaagtatgacaacacatacacacttgcctctccccatctccagaagaggggagcagagtccgagcttatctaaatatgaatgtggccacaaagctgtggaaggtgacaaagcttaaacacctttgccctggctctgcattgtcacctagagagcaagaggtctatagaaacatcatgtcacatgaaacgattctctgctttttggttctgaacttgaagtccctaaactgcaaaatctaagagttgggtggttattaaaatgcttttaaagtcaactgtggcaccaattctaatgtaatccaacttgtgactgtttttttttgttttgttttgtttttgtgtgtgtgtgtgtggcactgggaaaagtggaaacaaacatgtattgaaatacatattggaaataaaaatggtttgagcgtcagtgatattctcccagaatgtacttatcttacctcggcatgtactgtagtcactcagtatttgtatatgttgctagaatttagattgtaaaatagtgaaattttaatgtgttcatttgtttttaatgtatatatgtcttgctcagattatttggtttaaataaaacaaccttgaggtttgtagcttttccttatactata

SEQ ID NO: 19 - DNA, 2512 bp, Homo sapiens
agcgggcagacagtgcccgtgccggggcggggaggcgagttctccctgcggccgcggaccctggatggtggcgtcgcccgaagacgcgcctcaccggcctgggtggggttgccttttgccctcgaggctggcctaagcgctggggggccgctggctaaccctgaggtagatactcgggaaatgggtggaatagggaaagggactcagcccttccgcccttttccccagtcgaggcccgcgtcactgccctgtgggtgcagacgcctccagtgtcactcccctggatttcgcttgggcagaagaagagattgtaggccggaccccaacgccgaagccctgaggcttctaatgtgaggaaaaacacggaatgacaagtgtgggaaagagcaagcactaaattactgtcagaaaaataaacggagtagcacagtgctgctcggtgatttacgggggcaattagtcaccaccccacggcgctgctgaggacagcacggcagcagacaagtcaggcccatgaggaagaagaggctttgcactgctctccagacaacttttcaaacaacaagagaggatatttgagcacattaggttttgtgtcttcattctgggtacctactcttcatattattctaccaacttaatacataaacatatttttagcacaatatgaacctgttctatgtgtgtttaaaagcctccagcagcaacttttttctctcccactgtccaaagttgggttttccccatcccttaaaaaacaaaacaaaaaaacctttcttcactccaaattgtccccctggtgtgtggttttggtctttctcattacttcttgcatcttgctacatgttctgcatttgctcagagccactgggtgctaaggaatttggtctgctgctcctctgggaaagtttggtaagctggtacaaggaggtgatgaacaaggctaaatgaaaatcatacataaacttcccaatatttgtgaacatgttctttaacataccatacatagtttctatgtatatgtaacatagcaaaacaaacttgctaaggtataattaattggtatgcatgccttcataaatattcaccatcagaagaaacctagaatgaaaggttgtgaaagaacccaggaatgagaaagactttattgactaaaggccatgtctatgtaggggcaacaatgaagctgtgggtgagcaaatgaagcttcatcaggagtttagtatccttatgtagcattacctaagaatgctaccatagtaaatttctgaagtactttacctgttacaaagaacgtttgtaatctccttttcttatttgatcttgccctggctttagtcaggaacacctgctcagttagaaatgtattcggtgggtatgtacgcagttggagaatgtgccccaacacacgccgcctttggcctgtatgttggagtcctagagaggcagacaatgttgatgagcttggtaccagcatggtgcgccattcatccggacacaatttctgataaagagggtcacaactgggagaaacctcagattctacttgcttctcttctccacgttattagactttttttcccctcttgaattttataagcacactccttttaaaacaattaattaataacgagcactgggatttgatgtagaattgcgtatttgtaactgcaatggcactggttttcaactaccctgtaacaaggaagcaattctaaccagcaaaagaggcagacgtgtttctgtgcaacatcacaggcgtgtgaaattgttcagatattgagatatcaggagtgatataaattggaaatttaaccaaaaatattttccatcagtgaacaaagccgattttaaagtgcctcttgcatttttcagatctttctctttctttttcccaaagtcacattgtagtttctgataaaggaacacactgggaaaactaagcacatatttaatcaatgaaatttaaggtgtaaaaattctgcatttctgacaaatttaatgttcacttttttcttgatttaaatagagcattctaaaaatattacccctataggggataaatttttatatgcatgttatttttctttacaaagtgtgggctagtaaagagtgaaaaagaccttggctattgtacttatttcttgaaagaaataatagcatcttaatgactggattcaaatctctgcttatcacacaacttccgtacagttcaaagagaggacttggattgaatgaaaacagagagctggggcttcctacattcccctgtgtttgtgttcatacagaatatttatgcaatattctgacttggccataatctgtaattcctgcctattattctcagttgcaactataaattctagattaatggttataattttggtaaggcatacatgaaaagttatatgttcctgcttgaaaggaggatcattaaatattcattattaccactcaaaaaaaaaaaaaaaaaaaaaaaaaaaaaaaaaaaaaaagaaaaaaaaaaaaaaaa

SEQ ID NO: 20 - DNA, 4145 bp, Homo sapiens
cccaagctgcgccggctggcgggagagcagcacgcaaggacgccgaggtccgccgcgatcttccaggtgccctttgcccctgggcacagtatgacccgacctacagggagccctagcgcagggctcctgcaacggggcagcctaggataaaaaggatccttgccaagctcctaccaggccgcctttgagtctttaggaacccctcctccggctgcctccccaaggttctgggcctccttccctgcggcccagagccatggagctctccgatgtgcgctgccttacaggcagcgaggaactctacaccatccacccgacgcccccggccggcgacggcaggagcgcctcccggccgcagcggctgctgtggcagacggcggtgcgacacatcacggagcagcgcttcattcacgggcaccggggaggcagcggcagcgggagtggaggctcgggcaaagcctcggaccctgcgggcggcggccccaaccaccacgcgccgcagctgtcaggcgactcggcgctgcccctctactcgctgggcccgggagagcgagcgcacagcacctgcggcaccaaagtcttcccggaacgcagcgggagcggcagtgccagcggcagcggaggcgggggcgacctgggcttcctgcaccttgactgtgcccctagcaactcggatttctttcttaatgggggctatagctaccgaggggtcattttccccaccctgcgcaactccttcaaatctcgggatttggaacgcctctaccagcgctatttcttgggccaaaggcgcaaatcggaagtggtgatgaacgtgctggacgtgctgaccaaactcactctcttggtcctacacttgagcctggcctcggcccccatggacccgctcaagggcatcctgctgggcttcttcaccggcattgaggtagtgatctgcgccctggtggtggtcaggaaggacaccacctcccacacgtacctgcagtacagcggcgtggtcacctgggtggccatgaccacccagatcctggcagcaggcctcggctacgggctcctgggcgacggcataggctacgtgctcttcacgctcttcgccacctacagtatgctgccgctgccgctcacctgggccatcctggccggcctgggcacctcgctgctgcaggtcatcctccaagtggtcataccccggctggcggtcatttccatcaaccaggttgtggcccaggcagtgctattcatgtgtatgaacacagctggaatcttcatcagttacctgtcagaccgggcccagcgccaagctttcctggagactcggaggtgtgtggaggccaggctgcgcctggagacagagaaccaaagacaggagcggctcgtgctttctgtgctcccccggtttgttgtcctggaaatgatcaacgacatgaccaatgtggaagatgagcacctgcagcaccagttccatcggatctacatccatcgctatgagaacgtcagtattctttttgcagatgttaaaggatttaccaacctctccacgaccttgtctgctcaggagctggtcaggatgctcaacgagctctttgccagatttgatcgactggcccatgagcatcactgccttcgtattaaaatcctgggggactgctactactgcgtgtctggacttcctgagccccgccaggaccatgcccactgctgtgttgaaatgggtctcagcatgatcaaaaccatcaggtatgtgcggtcaaggacaaaacacgatgttgacatgaggattggaatccactccggctcggtgctgtgcggtgttttgggactaaggaagtggcagtttgatgtctggtcttgggatgtggatattgcaaacaaactcgaatctggaggaatccctgggaggattcacatttccaaagccacgctggactgtctcaacggtgactataacgtggaagagggccatggtaaagagaggaatgaattcctgaggaagcataatatcgaaacttacttaattaagcagcctgaggacagtctgctgtccttgcctgaagatatcgtcaaggagtcagtgagctcctcagaccggagaaacagtggggccacattcactgaaggatcctggagccctgaactgccctttgataatatcgtggggaaacagaatactctggctgccctaacaagaaattcaataaatctgcttccaaaccatcttgcacaagctttgcatgtccagtctgggcctgaggaaattaacaagagaatagaacataccatcgacttgcggagtggcgataaattgagaagagagcatatcaagccattctcactgatgtttaaagactccagcctggagcacaagtattctcaaatgagggatgaagtgttcaagtcaaacttggtctgtgcatttatcgttcttctatttatcacggcaatacaaagtttgcttccttcttcaagagtgatgccaatgaccatccagttctccattctgattatgctgcactcggctctggtcctcatcaccacagcagaggattataaatgtttgcccctcatcctccggaaaacttgctgttggattaatgagacctatttggcccggaacgtcatcatctttgcatccattttgattaatttcctgggtgccatcttaaatatcctgtggtgtgattttgacaagtcgatacccttgaagaacctgactttcaattcctcagctgtgtttacagatatctgctcctacccagagtactttgtcttcacgggggtgttggccatggtgacctgtgcagttttcctccggctgaactccgtcctgaagctggcagtgctgctgatcatgattgccatctatgccctgctcactgagaccgtctacgcaggcctctttctgcgttatgacaacctcaaccacagtggagaagatttcctggggaccaaggaggtatcactgctactgatggccatgttcctcctggctgtgttctaccatggacagcagctggagtacacagcccgcctggacttcctttggcgagtacaggccaaagaggagatcaatgagatgaaggagctgagggaacacaatgagaacatgctccggaatatcttacccagccatgtggcccgccatttcctagagaaggaccgagacaatgaggagctgtattctcaatcctatgatgctgttggggtgatgtttgcctccatcccaggatttgcggacttttactctcagactgaaatgaataaccagggagtggaatgcctgcgcttgctcaatgagatcattgctgacttcgatgagttgcttggtgaagaccgatttcaagacattgaaaagattaagaccattggcagcacctacatggccgtgtcaggcctgtcacctgaaaaacagcaatgtgaagacaagtggggacatttgtgtgctctggctgacttctcactcgccctgacagaaagcatacaggagatcaacaagcattcattcaacaattttgaactccggattggcatcagccacggctcagtggtagctggcgttatcggcgctaagaaaccacagtatgacatttggggcaaaactgtgaacctggcaagccgaatggacagcacgggggttagtggccggatccaagtcccagaggagacctatctcatcctgaaggaccagggctttgcctttgattaccgaggggagatctatgtgaagggtatcagtgaacaggaaggaaaaatcaaaacgtactttcttctgggaagagtccaacccaacccattcatcttgcccccaagaagactgcctgggcagtactccctggccgcggttgtcctgggacttgtccagtccctcaataggcaaaggcagaagcagctactcaatgagaacaacaacacaggaatcatcaagggtcattacaaccggcggactttgttgtcacccagcggcacagagcctggagcccaggctgaaggcaccgacaaatctgatttgccataaaagcattttctttctgttttttttttttgtatttcttttatatataaaataaatatactaataaaaaggtttaattttttttagaacaaaaaaaaaaaaaaaaaaaaaaaaaaaaaaaaaaaaaaaaaaaaaa

SEQ ID NO: 21 - DNA, 9215 bp, Homo sapiens
aaacagcgccgagccgccgccgcctcagcagcagcagcagcagcagcagcggcagcagcggccgtgcacgcccgggctgcggtcgcacagcgctaacgtgagcggccgccgccctcgccaccccgcctgcccactcccgccgccgccccgctctcgctttccccccggcctcccctcgccccttcccctcccccttcccggcgcactcggggggctgggaacgagctgccatgtgatgcgcgtcccctccgcgagctttcggtgacccacgaactgcccacctcgccggctgccgggagggggctgcgagccgggaagacgcggggaagaggaggcggaaaaggacgcaaagttctccggcgagcgcgttcgttcacatagctcccagttttaacatttcgccacctactgaagacatcatttgggaccaagctgatgagcccttgaagcacagcagataagagtgttgctgttgatcatctttgcctgggaagttgaatgataaagccagaagaaagcatgctttctgatcatttgcagctgtctgcttcagaaagtgagggctccaggaatgaggagaatcttcaagaacttcctcgcactgtgacatgtctgatcccttgctcccatccctgcagcatgaacaaggtggacactcactgacctgtcacaaggttgccccacaaaactttggggtccatgtctgaatggattgccagagccttctcatctctcccttcgcccagttccctgcatcctaagactcgaaggcagcacaggacctggaaaaattacatggtgtgaacggcatgtctgtggatgagaagcctgactcccccatgtatgtgtatgagtccacagtccactgcaccaacatcctcctgggcctcaatgaccagcggaaaaaggatattctctgtgacgtgactttgatcgtggagaggaaggagttccgggcccaccgggctgtgctggccgcatgcagtgaatatttttggcaggcgctggttggacagacaaaaaatgatttggtggtcagcttgcctgaggaggtcacagccaggggctttgggccgctgttacagtttgcctacactgccaagctgttactcagcagagaaaacatccgcgaggtcatccgctgtgctgagttcctgcgcatgcacaacctggaggactcctgcttcagcttcctgcagacccagctcctgaacagtgaggatggcctgtttgtgtgccggaaggatgctgcgtgccagcgcccacacgaggactgcgagaactctgcaggagaggaggaggatgaagaggaggagacgatggattcagagacggccaagatggcttgccccagggaccagatgcttccagagcccatcagctttgaggccgccgccatccccgtagcagagaaggaagaagccctgctgcccgagcctgacgtgcccacagacaccaaggagagctcagaaaaggacgcgttaacgcagtaccccagatacaagaaataccagcttgcatgtaccaagaatgtctataatgcatcatcacacagtacctcaggttttgcaagcacattccgggaagataactctagcaacagcctcaagccggggcttgccagggggcagattaaaagtgagccgcccagtgaagagaatgaggaagagagcatcacgctctgcctgtctggagatgagcctgacgccaaggacagagcgggggatgtcgagatggaccggaaacagcccagccctgcccctacccccacggccccagctggggccgcctgcctggagagatccaggagcgtggcctcgccctcctgcttaaggtctctgttcagcataacgaaaagtgtggagctgtctggcctgcccagtacatctcagcagcactttgccaggagtccagcctgcccttttgacaaggggatcactcagggtgaccttaaaactgactacacccctttcacagggaattatggacagccccacgtgggccagaaggaggtgtccaacttcaccatggggtcgcccctcagggggcctgggttggaggctctctgtaaacaggagggagagctggaccggaggagcgtgatcttctcctccagcgcttgtgaccaagtgagcacctcggtgcattcttattctggggtgagcagtttggacaaagacctctctgagccggtgccaaagggtctgtgggtgggagccggccagtccctccccagctcgcaggcctactcccacggtgggctgatggccgaccacttgccaggaaggatgcggcccaacaccagctgcccggtaccaatcaaagtctgccctcgctcaccccccttggagaccaggaccaggacttccagctcctgctcttcctattcctacgcggaggacgggagcgggggctcaccctgcagcctccctctctgtgagttctcctcctcgccctgttcccagggagccagattccttgccacagaacatcaggaaccaggcctgatgggagatggaatgtacaaccaagtgcggccccaaattaaatgtgagcagtcttatggaaccaactccagtgacgaatccggatcgttctcggaagcagacagtgagtcgtgtcctgtgcaggacaggggccaggaggtaaaacttccttttcctgtagatcaaatcacagatcttccaaggaacgatttccagatgatgattaaaatgcacaagctaacctcagaacagttagagtttattcatgatgtccgacggcgcagcaagaaccgcatcgcggcccagcgctgccgcaaaaggaaactggactgtattcagaatttagaatgtgaaatccgcaaattggtgtgtgagaaagagaaactgttgtcagagaggaatcaactgaaagcatgcatgggggaactgttggacaacttctcctgcctttcccaggaagtttgccgagacatccagagccccgagcagatccaggccctgcatcggtattgccctgtcctcagacccatggacttgcccacggcctccagtattaaccctgcgcccttgggtgctgagcagaacattgcggcctcccaatgcgcagtgggggaaaacgtgccctgctgcttggagccaggcgcggctccccccggacccccctgggcacccagcaacacctccgagaattgtacctctgggaggagactagaaggcactgacccgggaaccttctcagagagaggacctcctcttgaacccaggagccaaacagtgaccgtggacttctgccaggaaatgactgataagtgtacaactgacgaacagcccaggaaagattatacctagtgactcggctctgcctcccagtccgcacacctctcccatccaggcgttcttcagtcagcctgtggcactgttcatctgctgtcccgaagaaaccgagaacacatttggtgcacactacagcggtcttagcagcaatactgttccgaagtatcctctcctcttctcgagcaggagtgatagttaccttcacaatggtgctaccccttgcccaggcaaggaaagacagcagtgatgacactgtctgtctgtggctcaatttcagtcttcacagggatagactacaacacctctaggccccaaccacggattttttttctcagtggcccatgtcacaaaccctatctcaggaatttcttctgaatgttcaatttttttcattgaagacagcttctatacacatcaaagttttatagctagactgtacatattatatataatatatatataaaatatatatatatatatatatatatccatatgcaaaagtcctgcatgcctcaactttctcatcctaaaactggaaacttatttctcatttagaaacaggttccaacattcctcttcttttgtctctgatgctagaactagtttggtaactgttaacatggtcatttttcttgcttcacagttcaattttcaattcgtacttatttatggacaaaattcagtgttggaagctttttcccaaggttttatttcagatttctttttcgtttggtttggttttggcacctccaagtggtgtcatttgagcattgtaggtttgttttttgtttgtttggggggttttgtttgtttttgtttttgtttttgttttccttgcagatactgtacagtaatggtcaactttgccacttgcactgagttttgggtcaaacctattttcttaaatgaagttgtaacttcggtataactcaagtatactgtatattctttgcttttagttaaaaaagtaaaacattttagctaattaaaaagcactcaggtgataattatgtaggaaaaacaatcttgccaaataatgaattcatcctaggatgtgtagacaataatctgcttgaatatttttatatttcacctcctccccacctttccctaagcaaagtttaaacgcagatagagagttcagagttgatgctggatgttcagattcctaagtggggagagagtttggacatctcactcaaaagtacatcagaaaaacaggaatccgtgattttataccagaactcagcaggcattggctcctagaaatcaagttagaaagttttcacccagggagtaagtcccattcatttcaacacgtcctgaggcctcggcttgctcttggaagtgttgtgcagtaggacctgctcccctgaaggacggggccaaccagccactggctttcctgcccaggcttggcctcccaggacatctggcctgaggggatttgaatcacagccccgaaggtcctgccttcaccccattgggagagagcagggcatcctggcatctgcgatccatccctgacacaggctgacacattctttctcctttccttctccaaaggcttggagttttcttctgaggtttttctgccagtgtcttgtctgaaggcagacttcattctgaggctttggacaagctatcaccgggaaccctccctgtccccttcccgaatcacacacataccctaccctcacctgatgataattttctcttcttgctgcaaaactggttggcttgcaacccagagagagcagcttcccttggctctggggccgtgttggccccagccacgtttacaggaaggtgtgccccagaggaggaggaatcagctccctcgctccagtggccttgggtccgggtctcactgagcagcccgagggccactccagcccggctggggaagagagtcctgaacggtttgatgtggggatggggtggtgggcagtggggaatagatggttgactttgtttctttatttgtgccattgtttggacaatattaaagctgcatgtaaaaggggaaattagtatatgatgtaggctaaaagtgaaatcatagtaacatatgttttagtattattaacttttttctgtacaaatattagcactaaatgtttaaatatgtatgaatgccagaaatttgtcagttcatgcagtaggataaaaaaaaaaaaaaaaaaaaaaaaaaggcttttctttttaaacagttccacttttaaaacctgcctctgggtttttgttttttcttgtttgtgtgtgtgtgtgtgtgtgtgtgtgtgtgtgtgtgtgtgtgtgtgtgtgtctgaaacagatcttgataaagctctgtgttggagctgctggtttttgttatggttgttggaatttcttggcctactaggacagttctgtgcttcaccatgaggtttgcctttgtggaaaactggtgacagtgagaatataaactcaatgtgaatcacgtgatacttcgcaggcgtgtgttacagtggagtcagctgacagtattttgctttttaactctattgttgcctttccaagtgacctctcctcttcttttaaaaaaagaacactttctgctcatatcataaccaggtccaacccagcttcttggcatgaggtttaccctggtaacaactcatgtgcaactggtagtcttgaccacattccatccatttcctcaggtttctgtggttcagtagcccagacctgtttggcagccatttctagcaggggcggggcctctttatttctctccaccctaactcagacctcaccttcctcccacccacccctgccttgcttttcttcctcttcccccaacctaacttctgccatgggaactggttaaaaacactgctctaaaaaccatcttccaatttcatagagatttctcacaagttatttcattcataatccaccatgaacagtgactagcttcgtgcagttgttcatgtgatgtgtgtgtgtcttttcctattcagaactatgtgcttgtcaaaattatttctgggttgattcaaagggaggacttgctggggaccagaatccaaacggcctcaagtggaattttaaaacctagcctgtctcttttccctgggatccctctgtcaaccccacgccttttaggaaaaagaaaagtgagtgaacagcaaggaagagtgtttgcacagtacagtaacatttggttgttcttaaggctcttttcttacaaaaataagagaccctccaaccacgggctgtttaggaggatgcctgcttgggtctccaaatggctggggtaggaatggttgttggggcagagccagtggaggtgagtgaccctgagactaatgaacatcccacctaaatccagtcctccccttggatctgcctttgtcctgcttgtgtatccaggcaacctcttttcaagttggtcaggctttggacaggtgagtgatttgctgtatgtgtttgtttctctgcgttacctgggggtgccttgattaaaatcgaactttattacatactgattctggaacaaaacagttagaaaaactttaaaaaaaaaaaaaccgacaaagttacgaggccatcctgctatttatcttctgagttcccagcaatgactcaggcatcagagatgatgctgcagtggaaaacctgactctgtgtgtctgcaactgaatgttgtgcgagtaatttattaactgtctttctaaaggtttgctgcttttaagatgcactataattcgggatgtaatccttacattgcttttccaaggaagggaacaaaagtctagtgattagtatgccaactgccactactccttcaaaaggagccaggaccagcgacaagactcatgagaggactggctaaagtgaagtgtgcacagtgtgaagtttaatgctgttgtcaagaggcctaaacccacattttctcttttaatattttatgattgccatcaaagaagaagaaaaagaaggaacagacaaaggtttgaaaatgataagcctgttaagacaccaaaaactcctgtcccgtgaagctgcttgacatcctgtggagtagcataatcctctcaaaatgaggaagagctgcctgcaaagctttctcaagtccctatttggctacctacttctctacattatgccccatttaaactaggagctgtcttagaaatgacttcaaactgcttcactattgcttacagtttaggaggagtctcagatccagaaggagcaagaatcaagtttggtcctcaaatgactgtaaatagactaaagaacaaggtgttttttgtttttgtttttgttttctaagaataaagctgttcgttgtatcatgagttagtgtttcttccccaaactgaagactgtgttggaagtgcaatttctggtgagtcagtccacaatacaatgccctgtgtggagttggtattcatacaggaaatctgtgtgcacgaggcattgtgtgttgaaagtgtatgtttatagtactgcctgagccatctcatgaccccagcgtccaaaaccgatgctgtagaacagaacatatctgtacacaaatagtgtgtgcaaatagcatttgtacatagaaaagtctcattgtggcagattgagcataaattattcaactgacggtgcaaaaacattacttgcaaagaaaagtttatagtattttcctacactccaccctgggagatgatatttctatcaaatgaatatcagtgcattttaaatgtaatatgaaaacgatgctgccattttgtgaagaatacccacttggttgcagaggccaactttcatagctttgatttaatgttgtgacacggtgtatgcattttgctgtcaagcaatggataaacagctctgactttcattctcattccagtttattgacctcagataaaacactggcccttcttagaagcagaagtgtgcaccaagaccattcatttcaggtagactcacattcagtgccaagtgctcccatgggaataatcagacgcatatgttgcgaaagagtgaagggacttggacaaagaggggttttcctacagatggatgctcagtcttctaccaaaacatgtttggaggcagaactatgacctccccttaagtcctaacaatgtattttgtgtgtgcaaatcctgggatgcccgtttcacgctctgacataaagacatggcacctctagtgagtgatcaggaagattccatatgcatttgggagcttcaggtgcttgttagacacagtgagccattcaaggcaagcaccacctttgctagtgaggccaagagagcctgtgacaatttgacaatttgttccagaaccagtctgatgcaagtgcacctctaatatatgccttacaaactccagaggccatattcaaaacagggtcttctcagtgtatgcaaggggctgcagcccctcttctcttcctccccaggttgaacaatacggacagttttcacacatatctacctgtataaccctctgtacctctcataactggtcaacgactgtaacaggttacatcaggtgtttttctacatactttttacacagattctatgcgattaatgtaatttaattcaatgcatcattttattgtactagttcttaggcttgtccttatttttttctaagtgattgtggtttttctcgtggtttttattgtaaaaaatgaaaggctgttgatgcttattctctgtaactaagaatttttaccttttgggggaaaaaagcattgctatgaactaatgaattgaaacttcatttactcattgtaaatacactattgtgcaaaaaaagttttcactcaattgaattgctagtgttaactgaattttgtctagacaccatttctgttgatgaaataaagacatatcattatgcattgtaaactga

SEQ ID NO: 22 - DNA, 2568 bp, Homo sapiens
gccagggaccagtggtgggaggaggctgcggcgctagatgcggacacctggaccgccgcgccgaggctcccggcgctcgctgctcccgcggcccgcgccatgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctgagcacactgccagtctcactgtgggaaggccagctgccccagccagatggactccagcctgcctggcaggctgtctggccaggcctcttggcagtcacatctcttcctccgaggccagtacctttccatttattctttgatcttcagggaactgcatagattgatcaaagtgtaaacaccatagggacccattctacacagagcaggactgcacagcgtcctgtccacacccagctcagcatttccacaccaagcagcaacagcaaatcacgaccactgatagatgtctattcttgttggagacatgggatgattattttctgttctatttgtgcttagtccaattccttgcacatagtaggtacccaattcaattactattgaatgaattaagaattggttgccataaaaataaatcagttcatttaaaaaaaaaaaaaaaaaa

SEQ ID NO: 23 - DNA, 8320 bp, Homo sapiens
gtgtgtggatgtgtgagtgagagggaacgagagtaagagaaagaaagaagtgaggggatgtaaactcgaataaatttcaaagtgcctccgagggatgcaacgggcaaaaactgaactgttcaggcttcagattgtaactgacgatctgaggaaaaatgaggtgctcgatgaattttcgtttgtattttttggcgaggcgggggaggtgttgagattttttttttttcccctcggggtgggtgcgagggggatgcatcctagcctgcccgacccggagcaagtcgcgtctccccgccggagcccccccacccatttctttgctgaacttgcaattccgtgcgcctcggcgtgtttccccctccccccttccctccgtcccctcccctccccggagaagagagttggtgttaagagtcagggatcttggctgtgtgtctgcggatctgtagtggcggcggcggcggcggcggcggggaggcagcaggcgcgggagcgggcgcaggagcaggcggcggcggtggcggcggcggttagacatgaacgccgcctcggcgccggcggtgcacggagagccccttctcgcgcgcgggcggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaaaagggtccaagttgccacattgcttcattaaaacaagagaccacttccttaacagctgtattatcttaaacccacataaacacttctccttaacccccatttttgtaatataagacaagtctgagtagttatgaatcgcagacgcaagaggtttcagcattcccaattatcaaaaaacagaaaaacaaaaaaaagaaagaaaaaagtgcaacttgagggacgactttctttaacatatcattcagaatgtgcaaagcagtatgtacaggctgagacacagcccagagactgaacggcaatctttccacactgtggaacaatgcatttgtgcctaaacttcttttggaaaaaaaaaatataattaatttgtaagtctgaaaaaaaaatatttaatttaaaaaaaattgtaaacttgcaataatgaaaaagtgtacttctgaagaaaactacatgaacgtttttgttggtattcaagtcagctagtgtttataattactggatattgaattaggggaagctcggctgccctagtaacaaaaccagcaaacgtcctgatgacaacgaagtgatgacattagccattccttagggtaggaggaacagatggatcttatagacctatgacaaatatatatataaatatatatataaatatatattaaaaatttagtgactatggtaagcttttgttcatttgtttcagacttttttctcctgtaaaaaaatagtactgattaacttttttaaaagaaagattttactgtaaatatggatttttttttttttggtcttatttctgtccctttccctggtttgttatcgtaacctgtagtgccaactctgcttccagaggggtagtgcaggatgaaatgctgaccctgatgttgcttctcattcataaataagtagaaagttgtttctccagtcttttgggaacacaggacttaaaagtcacatcatgtgtagatattacaagcagcattaccaagacatggcaaaaagagtttgtctgaattgtaatgttgcgtttgtgaacctattctgggattttcagaggtacaaggttagaatgctacaatgttaccactgtgccttccaatgtttatatcatcggaaacataacataatcaaagtggctgtgatttaacaaaatgattaaagtgttacctacctgtgtagccgaagtagtgtgcagtgaggcgtttctgaatacatggtcagatttttggaaaaaaacaaaaacaaaaaaaacaagtaaagttcaaaaaccgtcaaatgagaaaattgcaagtagtgtgacagagctgattgattttgttgctttcttgattttttttttcaaaatgggtttactaaaatgtagatgacttaactgcctcctccttcgtctgaaaaatgccaatattcaatcatcatgcagcattataacaagccttataagtcctaaagcattaagttgcacttttttgaggaggggtagtgcagtatttctctggccagtatgaatgaagtttatacttaccatatttgatagaaacatagatcaagctatggcacagcgactcatcagatagctagctttgacgtctgggcacaattgaaccaacttccatcgtgaatctttataatgattgactttggtgtatagtgcagtaaacaaatagtgctcctagttaagtatttgtcagcatccttttgtctctaacttgtttctatttttacagccacacaattcttggcatgtattaagaaaaaaaaaaatccctgttcaagtagtttttccacctatcagcactgagtaaatgccataaatccattgaaatggtctaaatgttccatctgttctcctgttttgccagttatatagtaatgaaatacatttgtaaattttatgcaacaaatggcaaacgtatcattattttgaaattgtgtatgtaaaagttatatttttacatgtagactcttgttattatgtgttttaatacattgtatcagtttttgtttttttttaaaaactgtggtttaaaaagaagtctcatttaaatgaaatagctacaagaatcagaattttatgttcatttctgaaaatgtaagaacaaataagatagttaccacgtggtcatcttttacaaacccataaacattttgattagctgtgtgtgtgttgaaaaactgtaaatatgttcagtagcgataaaactaaaataactttgatttgttgataagttcctaaaatgtggaggtggattaaaaccttaggagaatagcagaaatcaaacttcatgaaaagttattttggggctttcctgtgaaatgtatgaacaaagaggctcagagaaggacatggaagacaataatgtatactctctcctcctccctgaataatgaaaaccatgtgtatttgttccctccgtatgttaaagatttccttttagtggtacattctgcactcattttgtatagtctaccaaggcgggtatccctaggaacaatattatataggaagcaggtatactctgatcacattcaggataagtgtacagaagaaaatacggtgtttactctttagggaactggaaacactccctgcattgatgtacattttaagaatggcacttttgatacatgttatcataaaggtgcttaatagagctgaattaaagtttttcaaatctgtaaacaaagcaaaaaagtaaattgtagtcatttgattattttttaaattggtgctttatattttgttctcactcagagtaaaagctgcaatttattgttcaccagctttgatgtattcattactcagtaatgcaatacctctattgttgaattccctttggaaataagtgaaaattctaacggccactgaaagctgctcgctaggttttgcttggtggagaaacataatctgcacctatccatattaattgggttgtatccccattaaaaaagaaaaaaagggaatgtggcctttttagtgtgttttttattgttgttgttttgtaattatcaaacccaggtaagatattggtatcctgcactggattttcaaatgaagttcagcagaagacagttaagattaaagtactatacaaaaatttcaaaagggtccatactacgctatctgtatgacgacacttaggctggggatctctttcagaaactcggactttaaaagcaacttggagcagttgatccacctccacattcaagtaatttatgaatatgcagaatagggatctgttcatctagaaatttttaccatttgtcttctgtgtagctgcaaggaacactaatgtttatacaactgtcagtccacccagtggtgcaactggttctgattcagtcttccgattcctttttatttttcactttttcctatttctgaatttttttttttatttgtgatcttgattttgatgaggggttggggagtggggagggagtcgaaccaagacttggagttaagaggattttcatcttttgcatccaacaggcagaatatgatctgtgtccaaaagtgaacttgagtcaggaatgaatcaatttcagcataaacaagcacaaaaatttagtctgctggctgactggaagcaaaaaagtcaagatggaatatgatgaattccaacacaatggggcaccaaggcctttaggcctctctttttattttgctttggttttgtttgtttttctttagagacatgctctttctcatgggacttgaagtggactcatctttgtgcagtgctggttttgccatactcatttcaagtattatagacatatgtaatggtgaaaatatatgaactgtggcctttttcattcttgttacttgtgatgcaattaagtgaagataagaaaaaaaaaaaaaaagcagagatttaccatgtatcagtgcctggctttttgttataaagctttgtttgtctagtgctcttttgctataaaatagactgtagtacaccctagtaggaaaaaaaaaaaactaaatttaaaaataaaaaatatatttggcttatttttcgcaggagcaatccttttataccatgaatattacaaaaaaattgtcagattctgaatatttcttctttgtagatttttggaatcattatgagtaaaagtttgttactttattttactatttaaaagatgttattttaccatgtgttaccaagatgaaactgtatgggtagcttttttgtttgttttttgttttgtttttgtttttgtttttgtttttagttgtaggtcgcagcggggaaattttttgcgactgtacacatagctgcagcattaaaaacttaaaaaaattgttaaaaaaaaaaaaagggaaaacatttcaaaaaaaaaaaaaaagataaacagttacaccttgttttcaatgtgtggctgagtgcctcgattttttcatgtttttggtgtatttctgatttgtagaagtgtccaaacaggttgtgtgctggagttccttcaagacaaaaacaaacccagcttggtcaaggccattacctgtttcccatctgtagttattcgatgaagtcatgtacatgaccgttctgtagcaataaatgtgccatttttataaactgtttctgacacttgtttcatttcattttgcattgtccatatagctatgattctcttctgtaagtaaaacgcatctatatttcattttccaagtgttggaggtattgacagcttaacaaacaaaacatacaaaaaaaatcacaaaaacaaattgaaaagcaaagcacatgattgatcaaggaagagatgcccttaatgaaaatggaacgggatgcatgcaaaacaaaaagaaaactgtctagaggattaactaattgaaggaatataattaatgtgtgtgtaacactgaagctatgcatttgaagagctctgaactgcaccagtgttttcggttgtgctgcaggttgctaagtcaagtcagccttaaccttttgcaccagttggtcggctgtttggcagaacattctcagatcttttcagtcaaaaatctaagatgatttattttgtatcactttgttaaaagctgaatattgttaactacagttaatattaacactgtatttatactttctcaaactacatccgccccaccacttctggttgcctctgttgactattaatccagatgtaaacaaccagatgtttttttctaacttgtacaaactgacgtgtgtcaactatcatggaaggaaaaaaatgtacagattaaaattattcagtgttatgtactgtaagttaatatttttgtagaatggacatcaatctactttgcaaaatttggaggctatttcaacattgcactgtagaaatgtaaagtaatgtatgcaatgtaaaggaaagcccgcggtagctgagcgcttcataacagaatgttctaatcaagtacgtggtatttggggatgtctccaatattgctcttgtattctttctaattgggtttagtgactagttgaaggaaaatgttataacgccatttggttcacatgtgaagtgccctccatagccaaatgttgggatttttttttttttcgtttttggttggactgtttgcagatatttaaattttatgaaatttccaaagattttggttgataacccccttttaccttctaaatgatttgagatgttcttatgttcttactgtgtgttttaaatatatataaaagagccacaagcattt

SEQ ID NO: 24 - DNA, 761 bp, Homo sapiens
ggcggcaggaccagcatgcaccaccgaaacgactcccagaggctggggaaagctggctgcccgccagagccgtcgttgcaaatggcaaatactaatttcctctccaccttatcccctgaacactgcagacctttggcgggggaatgcatgaacaagctcaaatgcggcgctgctgaagcagagataatgaatctccccgagcgcgtggggactttttccgctatcccggctttagggggcatctcattacctccaggggtcatcgtcatgacagcccttcactcccccgcagcagcctcagcagccgtcacagacagtgcgtttcaaattgccaatctggcagactgcccgcagaatcattcctcctcctcctcgtcctcctcagggggagctggcggagccaacccagccaagaagaagaggaaaaggtgtggggtctgcgtgccctgcaagaggctcatcaactgtggcgtctgcagcagttgcaggaaccgcaaaacgggacaccagatctgcaaatttagaaaatgtgaagagctaaagaaaaaacctggcacttcactagagagaacacctgttcccagcgctgaagcattccgatggttcttttaaagcagtagtatatcttattttcaaggcatttggaaatgaagggcaaactaatgtcttgttttaagaaactgcttagtccaccactgaagaaaatatccagaaattattttcattttatgtatagggatttcttcaaaaaaaaaaaaaaa

SEQ ID NO: 25 - DNA, 1360 bp, Homo sapiens
cggcccgggaggcggaggcgcgggggaggaggccccgcttggctcctcagccccggatgctgcatgacttcatccttccgccggctcccctgctgaggtagggccggtccggcagcaagcccgccgcccgcgccccgccgcagtcccgctcccgccccgcgcccaccccgcgcccgccatgtccgagatcctgccctacagcgaagacaagatgggccgcttcggcgcagaccccgagggctccgacctctccttcagctgccgcctgcaggacaccaactccttcttcgcgggcaaccaggccaagcgaccccccaagctgggccagatcggccgagccaagcgagtggtgatcgaggatgaccggatagacgacgtgctgaaggggatgggggagaagccgccgtccggagtgtagacgcgccggctcgggcggcgggctccgggcccagcctcgcagcggccaggagcgcgggccggcgatgcggctgccggcgcccccccgccccggcccaggcgcccgcgggcgggggctgcagggccgtgccgccgccgccgccaggcactccggagctgtccgcttcagcaccacggcggccgcggtagcggcggcgcggacccgcccggaacccgccgccgcgctcatgcactttaaaacctcgggccgcagccccaccccgcacaccggaacggacacggagacccgcggcccccagcccccggcccgccccctcccgcacggctcccctgccccgcccccctccggccagtctggtctgcagaccgggcggccgccggtgtcggaccgcgcgcggccagggatgtttggctgcgtacttgcatgagctttccacccacttgagcccagtcctccgggccgttcctcgcccccacccctgtctggcgtgaccccagcctcagcctctttctaagggacttcctcagcacatttgtatttttatatccgactctttgtttactggctctgctcatggttcctgccctcccctcagtctcggccacgctcttctgcgcctggcagataggatgggtgttgaggctgggatgagacaagcccccgacacggttaccaaccacatggccagtctgcccaggcctgaccccaggaggctttctggcagaccccttctccccaaggtatcatcaccttccaagggcccagatccgattttaccttggacccctgcgtcttgcccctgggaacccaaatggggactgctctgaacctttggatgacatctgtggccccggagatgttctcaacccaggggtgcccttcgtaaatgttcctccatcctcactgtaccaggagtgtgtgaataaacacagaccccctctg

SEQ ID NO: 26 - DNA, 2188 bp, Homo sapiens
aggtgagcggcggccaatgggcgagcgcggggcaggtgcccgctaactcgcgcctcgcagcgctgggcggccggggctgggcagggcagtgcggggacaccgggggctggggtcggtcccagcgggactccgaaaggagggagacgagctcaaccctcgggccttactggcagctcgcagcctagcacggagcccgcgcctgtgcgggcgcctggagctgcccgctccgccgcagcagccgccgcgcctggccgtacgctgtggccggaccccgcggtcgctcgctcacacacccctcgccgctccgcgcctggctcgcccgcgggggccgagcgcgagcgggcgggcgggggaggtgaggggtgcgggcgggtgtgcatgtgcctggctgggtgcacaccccgcaaggcggcggcgccaggacgcggagcgctccccagagcccggctgcctcgcacagctcccgcggctgcgaccatgttccagcccgcggccaagcgcggctttaccatagagtccttggtggccaaggacggcggcaccggcgggggcactggcggcgggggcgcgggctcccatctcctggcggcggccgcctccgaggaaccgctccggcccacggcgctcaactaccctcaccccagcgcggccgaggcggccttcgtgagtggcttccctgccgcggccgccgcgggcgcgggccgctcgctctacggtgggcccgagctcgtgttccccgaggccatgaaccaccccgcgctgaccgtgcatccggcgcaccagctgggcgcctccccgctgcagcccccgcactccttcttcggcgcccagcaccgggaccctctccatttctacccctgggtcctgcggaaccgcttcttcggccaccgcttccaggccagcgacgtgccccaggacgggctgcttctgcacggccccttcgcacgcaagcccaagcggatccgcacggccttctcgccctcgcagctgctgcggctggagcgcgccttcgagaagaaccactacgtggtgggcgccgagcggaagcagctggccggcagtctcagcctctccgagacgcaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctgtatcttgagggaggacaggcccaggtctttcttaacgtattgagaggtgggaatcaggcccaggtagttcaatgggagagggagagtgcttccctctgcctagagactctggtggcttctccagttgaggagaaaccagaggaaaggggaggattggggtctgggggagggaacaccattcacaaaggctgacggttccagtccgaagtcgtgggcccaccaggatgctcacctgtccttggagaaccgctgggcaggttgagactgcagagacagggcttaaggctgagcctgcaaccagtccccagtgactc

SEQ ID NO: 27 - DNA, 1303 bp, Homo sapiens
gtgggacgcgcgcggctgtgagcctgcgggacatgccccccgcgccggctccttgctggcggccatgaagaggcagaacgtgcggactctgtccctcatcgtctgcaccttcacctacctgctggtgggcgccgccgtgttcgacgccctcgagtcggaccacgagatgcgcgaggaggagaaactcaaagccgaggagatccggatcaaggggaagtacaacatcagcagcgaggactaccggcagctggagctggtgatcctgcagtcggaaccgcaccgcgccggcgtccagtggaaattcgccggctccttctactttgcgatcacggtcatcaccaccataggttatgggcacgctgcacctggcaccgatgcgggcaaggccttctgcatgttctacgccgtgctgggcatcccgctgacactggtcatgttccagagcctgggcgagcgcatgaacaccttcgtgcgctacctgctgaagcgcattaagaagtgctgtggcatgcgcaacactgacgtgtctatggagaacatggtgactgtgggcttcttctcctgcatggggacgctgtgcatcggggcggccgccttctcccagtgtgaggagtggagcttcttccacgcctactactactgcttcatcacgttgactaccattgggttcggggactacgtggccctgcagaccaagggtgccctgcagaagaagccgctctacgtggcctttagctttatgtatatcctggtggggctgacggtcatcggggccttcctcaacctggtcgtcctcaggttcttgaccatgaacagtgaggatgagcggcgggatgctgaagagagggcatccctcgccggaaaccgcaacagcatggtcattcacatccctgaggagccgcggcccagccggcccaggtacaaggcggacgtcccggacctgcagtctgtgtgctcctgcacctgctaccgctcgcaggactatggcggccgctcggtggcaccgcagaactccttcagcgccaagcttgccccccactacttccactccatctcttacaagatcgaggagatctcaccaagcacattaaaaaacagcctcttcccatcgcctattagctccatctctcctgggttacacagctttaccgaccaccagaggctgatgaaacgccggaagtccgtttaggggaactaactgcacattcaagagaggcgtccgtggatgctgggtctcactgccaaagccgaacacggcttcgggatttctgccttctcaagtggacctctgctgtgctgggcg

SEQ ID NO: 28 - DNA, 3699 bp, Homo sapiens
gctgccgccgcggcggccgctgctgctgctgcttctgccgccgctgccgccgccgctgcctggatatagtgcggcaagagcggagcttgcagtcactttgcgaggaggagcgcgcgggctgcgggcggctggggcaccgcgggagcggcggcggcggctctagcagaggcggccggggcagcgaaaggttctctctccagggctggacttaataactttgaaactgtccaccggtgtcacgtcctgaacatgagcctcctcctctccttctacctgctggggttgcttgtcagtagcgggcaagctcttcttcaagtgacaatttcacttagcaaagtagagcttagtgttggagaatctaaattcttcacatgtacagcgattggtgaacctgaaagtatagattggtataatcctcaaggagagaagataatttcaacacagagggtagtagtgcaaaaggaaggtgttaggtcacggttaaccatctacaatgcaaatatagaagatgcagggatatatcgttgtcaagcaacagatgccaaaggacaaacacaagaagctacagtagttttggaaatttaccaaaaactcactttcagagaagtggtatctccacaagaattcaaacaaggagaagatgcagaagtggtttgccgagttagcagttcacctgcacctgctgtcagctggttgtatcataatgaggaagtcaccactatttccgacaatcggttcgctatgttagcaaacaataacctgcagattctcaacatcaataaaagtgatgaaggtatatacagatgtgaaggaagagtggaggccaggggagaaattgacttccgtgatatcattgttattgttaatgtgccgccagcaatctcaatgcctcagaaatcttttaatgccacagcagagagaggagaagaaatgacattttcctgcagggcctcaggctctccagaacccgccatctcctggttcaggaatggcaagctcattgaagaaaatgagaagtacatattgaaagggagcaatacagaactcactgtcaggaacataatcaatagtgatggtggtccttatgtctgcagggccacaaataaggcaggagaagatgaaaagcaagctttcctccaagtctttgtacagcctcacataatacagcttaaaaatgaaactacatatgagaatggtcaagtcacactcgtatgtgatgcggaaggggagcctattccagaaatcacttggaaaagagctgtggatggcttcacgttcactgaaggcgataagagcctggacggccgtatcgaagtcaaagggcagcatggaagctcatcactgcatattaaagatgtgaagttgtcagattcagggagatatgactgtgaagctgcaagcagaattggagggcatcaaaagagcatgtaccttgatattgaatatgcccccaagtttatatcaaaccaaacaatttattactcttgggaaggaaatcctatcaatataagttgtgatgtgaaatcgaatccaccagcatcaattcactggagaagagataaattagtcttacctgctaaaaacacgaccaatttaaagacttatagtacaggaagaaagatgatattagagattgcacctacatctgacaatgactttggacgctataattgcacagccactaatcatataggaacaagatttcaagaatatattcttgctttggctgacgtgccatccagtccctatggagtgaagatcatagagctgtcgcagaccacggccaaggtttccttcaacaaaccggactcccatggaggtgtacctattcatcactatcaggtggatgtcaaagaagtagcgtcagaaatctggaaaattgtacgctcccatggagttcaaacaatggttgttttgaacaacctggaaccaaatacaacttatgaaatcagggttgcagctgtaaatggaaagggacaaggagactacagtaaaatagaaatcttccaaacattaccagttcgtgaaccaagtcctccatccatacatggacagccaagcagtggaaagagctttaaactcagcatcaccaaacaggacgatggaggggcccctattttggaatacattgtgaaatatagaagtaaagataaggaagaccaatggctagagaaaaaagtgcaaggaaataaagaccacatcattttggagcatctccagtggaccatggggtatgaagttcagattacagctgccaatagattgggatattctgaaccgacagtttatgaattcagcatgccaccaaagcccaacattattaaagacacgctgtttaatggtcttgggcttggagcagtaattggcctgggagttgctgcactgctgctaattcttgtggtaacagacgtcagctgcttctttattcggcaatgtgggttgctgatgtgcatcactaggagaatgtgtggaaagaaaagtggctccagtggcaaaagtaaagaactcgaagaaggaaaagctgcatacctgaaagatggatcaaaagaaccaatagtggagatgagaacagaggatgaaagagttactaatcacgaagatgggagcccagtaaatgagccaaatgaaaccacaccactgacagaacctgaaaaattgcctttaaaggaagaagatgggaaagaagctctaaatccagaaactatagaaattaaagtttctaacgacatcattcaatcaaaagaagacgacagcaaagcataacaacaatattacaggggcttgaacaacactacgaagagtatttggattgcgtgaccctatgaccaaaactattccattgaccttaatttcttgggaaacttctagcttggaatagcttgtacacatatacatatgatcaaatactcctgcccatgatccattcccttttgttattgttgttgttgttgctgttgttgttaattttgttaagaatttcaatatcaagactgactggcaccaacactttggtattcaatttgattctatgactgaagtactggaatttattatgtggctaaagtgctctatttattaagaactatatttaataccaccaacaaatataggggttaaggaaaaaaaacgtgagctacatgtgtaagaaggccctgcatgtgtatgagtcctattctgggcaaatagattcttaaagtggctttcaacttcaagatgaaggagcttaataatggttactcattttatcaggggaatttcagggaacgtaggcgtcaaagagccagttatctttagcagatattaaaaattgaaaactttggagaactcatttcaagttatgattcagtgcattttcaacattgatttttgatagactgaagtgccagatcaaaattgttacccatttgaaagaatattagttgtatataaaattagattagaaagactttctaaatctctatctctttatatatgtcctattcattcacaatggattatacaaaaaaaagtgtattgcaagtgaaataatattgatttctgccctcagcttcaaataaagtaaattgaaatgggaacaatatcaatatggtgtcttgatatatttataaatatgtgattatcatttatttttaaaataatttatcaaaaaacaagtctttagtgttcaaatacttcaaatcatatcctcagatatatttt

SEQ ID NO: 29 - DNA, 4371 bp, Homo sapiens
cccctgctgctctcaagtttcccgttggcggcgcggcccgggcgcttcaggtagcctctcggctctctctgctccgctccgcgcccaggtagggcaccgacgggggctgcacgcggctggccggcttcctccctgctgaggcggccctccctcctcccgcggggccctcctggccggggatccgcagcgctgcgccctctgaacgcccggcccccgcgcctgctgcggggcgcggcctggccgggccctggccccggctggcctcagtgcccagcagccccgctccgctctgcccagcgcgtcccctttgctccagccctgcggccgtccctttcggccggcggcatggccctgtcgtccgaacccgctgagatgccgcggcagtttcccaagctgaacatctctgaagtggatgagcaagtccggctcctggcggagaaggtgtttgctaaagtgctccgagaagaggacagcaaagatgccctgtccctgttcactgtcccagaggactgccccatcgggcaaaaggaagccaaggagagggagctgcagaaggagctggcagagcagaagtctgtggagaccgcaaaaagaaagaaaagtttcaagatgattcggtcccagtccctgtctctgcaaatgccgccacagcaagattggaagggccccccggcagccagtccggccatgtctcccacaacccctgtggtcactggagccacttccctgcccacgccagcaccctatgccatgcctgagttccagcgggtcaccatcagcggagattactgtgccgggatcactttggaggactatgagcaggcagccaagagtctggccaaggccctaatgatccgggagaagtatgcgcggctcgcctaccaccgcttcccgcggatcacatcccagtacctgggtcatccgcgggcggatactgcacctccggaagagggccttccagacttccaccctcctccactgccccaggaagacccctactgcctggatgatgcaccccccaacctggattacttggtccacatgcaggggggcatcctctttgtgtatgataacaagaagatgctggagcaccaggagccgcacagcctaccctaccccgacctggagacctacacggtggacatgagccacatcctggctctcatcaccgatggccccacgaaaacctattgtcaccggcgactgaactttctggaatccaagttcagccttcatgagatgttaaacgaaatgtccgagttcaaagagttgaagagtaacccccaccgggacttctataacgtgagaaaggtggacacacacatccatgcggccgcctgcatgaaccaaaagcatctgctgcgcttcatcaagcacacataccagacggagcctgacaggactgtggcagagaagcggggccggaagatcaccctgcggcaggtgtttgacggcctgcacatggacccctacgacctcactgtggactcactggatgtccacgcgggccggcagacattccaccgctttgacaagttcaactccaaatacaaccctgtgggggccagtgagctgcgtgacctgtatttgaaaactgaaaactatctgggaggagagtactttgctcggatggtcaaggaggttgcccgggagctggaggagagcaagtaccagtactcagagccacggctctccatctacggccgcagtcctgaggagtggcccaacctggcctactggttcatccagcacaaggtctactctcccaacatgcgctggatcatccaggtgccccggatttatgacatatttaggtcaaagaagctgctgccaaactttgggaagatgctggagaacatcttcctgccccttttcaaggccactatcaacccccaagatcatcgagagcttcacctcttccttaaatatgtgacggggtttgacagcgtggatgatgagtccaagcacagcgaccacatgttttccgacaagagcccaaacccggacgtctggaccagtgagcagaacccaccctacagctactacctgtactacatgtatgccaacatcatggtgctcaacaacctccgcagggagcgcggcctgagcacgttcctgttccggccgcactgtggggaagccggctccatcacccacctggtgtctgccttcctcactgctgacaacatttcccacgggctgctcctcaagaagagtccggtattgcagtatctctactaccttgctcagatccccattgccatgtctcctcttagcaacaacagtttgttcctcgaatattccaagaaccctctgagggaattcctacacaagggactgcatgtttctctttccaccgatgaccccatgcagttccactacacgaaggaagcacttatggaagaatatgccattgcagctcaagtgtggaagctgagcacctgcgacctgtgtgagatcgccaggaacagcgtgctgcagagcggcctctcgcatcaggaaaagcaaaagtttctgggacaaaattattataaagaaggacctgaaggaaatgatattcgaaagacaaatgtggctcagatccggatggcattccgatatgagaccttatgcaatgagctcagcttcctgtctgatgctatgaaatcagaagagatcaccgccttgaccaactaggtccagcatttgacatgcattttaactttttggttcaatttcaagtctgctgtggctaatagtggtcaagattccgaactaggactttcctctgtgaagaggatgcctctgaagaaattttaaactggtgattttggttgcactgctcactttaagagttaacatgctcacttgttagtatttctgagtaacaagatggtgacttctccttggggatctgggagctgagcacttgtctatacttgttcctaattttccaagtatttctcttgaaactgccagtgcctgaactgttggggccaggattttccctggtcagatgccaagtaacatgtggttttctgccatacttttctccattggcccaggtaggctaattggtagttgttcatttcagcctctggatggctggctgccttaaacacaatcaatttcaaagctccatttcataaaggggctactttgaaggagttaagatggaagacttccttcttgacaaattgtgtttttagtgaatttcttaaaccgttttatttagccctccttccctctttctagttggaagccaaatgtactcatgaaaacagccactcctattctgagtcttggtttcttcacctagaaagtgagggtttggactagatgagtggctttcagggtgttctgtgaatctcctcatgaatactttagggtaggggagggaagggagtgagtgatgctcaggggctgtcaaagtgactgcgttcatcagttttacactggggctgctacataatattttcatttgaacgaagaacttcaaaaagcacaggactagatgatctctgttccttttggctctaatatgctacaactgtaggccaattatcactttaccaattaagagttaggccagataagtgaaatttaacttaagggcacacagctaataagtaataggcctaaactggatttccttattccaaatcctgtcttttccccactattccattagaccccacaaatgttagttgtgtgtgtgtgtgtgtgtgtttttaatcactgtaaccggatgcatttttttaaggcaaaatttctcccttatctactatgatgacttcagaagatacaatggtcccaggggccaagtagaaagcatttttaaagattaatctgaattaagctttatcagtgtactctttatctgtgttactagtgcctggtatgtagtaggtgctcaataaatgcatattgaataactaagtgaatttcttttggcaactttttaaggacatgtgctcttagtacttaagaggctgctcaaggaccttcctatctatttctgtggtcaaattcagactacaggaggtctttttgaatcattatagttagaagaagaatccagtttctgcctgtgaacttatttgactttagattgtctcatcttgtgactttataatgcctgtcccctccactggtcaattcagcatatggaagtataaatgcagtctttttactaggcagatatatatgctatcttacagctaattatgaaattgaatgaaaatccattgttatcttagggattagttttgaaaagcccccgtttatatacatttgccctaacataggaagtattgtgtagtttctcattgtcattttatctgtatcaagtatctttttttaaattgttaaataaagtcagctaggactgtg

SEQ ID NO: 30 - DNA, 1892 bp, Homo sapiens
aaaccggtgccaacgtgcgcggacgccgccgccgccgccgccgctggagtccgccgggcagagccggccgcggagcccggagcaggcggagggaagtgcccctagaaccagctcagccagcggcgcttgcacagagcggccggacgaagagcagcgagaggaggaggggagagcggctcgtccacgcgccctgcgccgccgccggcccgggaaggcagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaagttaccggcttctactgtatttttaaaaaaaagtaagcaaaagaaaaaaaaaagaacagagaaaagagagacttattctggttgttgctaataatgttaacctgctatttatattccagtgcccttcgcatggcgaagcaggggggaaaagttatttttttcttgaagtacaaagagacgggggaacttttgtagaggactttttaaaagctattttccattcttcggaaagtgttttggttttccttggacctcgaagaagctatagagttcaatgttattttacagttattgtaaatatagagaacaaatggaatgactaatcattgtaaattaagagtatctgctatttattctttataatatcccgtgtagtaaatgagaaagaagtgcagagcaggatt

SEQ ID NO: 31 - DNA, 3794 bp, Homo sapiens
gactcagatctcacctcctaccactcccctaggagagctgggggccactgtttcctggattatcctaaaagcttctgaggccgtgaggacttggcagcatccctgctccctccttcacctccccctttggcactgcctgtcacctcctttataaagcctggctcttttatcaccgccacttggccctcactgccgccgccagctctgggctccatggactggtcccgtctgaggtgcccctgaccgtccctgccctcaccccaccccggatcccggcaatgctaaccgctgtctgcggctctctgggcagccagcacacggaagcgccgcacgcctccccgccgcgcctcgacctgcagcctctccaaacttaccagggccacacgagccctgaggccggggactacccctccccgctgcagcctggagagctgcagagcctcccgctgggcccggaggtggacttctcgcagggctatgagctgccaggggcctcctcgcgggtaacctgcgaggacctggaaagcgacagtcccttggccccgggccccttttccaagctcctgcagccggacatgtcacaccattatgaatcgtggttcaggccgactcacccaggcgcggaggatggctcgtggtgggaccttcatccgggcaccagctggatggacctcccccacactcagggcgcgctgacctcacctggccacccgggggcgcttcaggcgggcttggggggctacgtcggagaccaccagctttgtgccccgccaccccacccgcatgcgcaccacctccttccagctgccggagggcagcatctcctagggccgcccgacggggctaaggccttggaagtagccgccccggagtctcaagggctggattccagcctggacggggcggcgcgtcccaaaggctcccggcggtcggtgccccgcagctcaggccagaccgtctgtcgctgccccaactgtctggaggcggagcgactgggggctccatgtgggcccgatgggggcaagaagaagcatttgcacaactgccacatcccgggctgcgggaaagcctacgccaagacgtcgcacctgaaggcgcacctgcgctggcacagcggcgaccgtcccttcgtgtgcaactggctcttctgcggcaagcgcttcacgcgctcggacgagctgcagcgccacctccagacccacaccggcaccaagaagttcccctgtgcagtctgcagccgcgtcttcatgcgcagcgaccacctggccaagcacatgaaaacccacgagggcgccaaggaggaggcggctggggcggcctcgggagagggcaaggccggcggcgcagtggagccccccgggggcaaaggcaaacgcgaggccgagggcagcgtggctccctccaactgagctcctcagtgccgcctccctgcgggtatcccggggggcactggatgcgagcccccaggtctgacgtccttgggggtggcttgaggaagaggggaaggtgcgtatttattcagggaggaggaaaagtggtgcagggacagggagatggggcgctaggggttcttagtctctggggctactaggcaggatgaatttgactgggtcggtaggagctgcgcaatgcccctctgttctcccctgcctcacagtttccctcgcccctgggctggggggttggggtgggacacccgtaccgcggctggctggcggggacaggctagaggagacagcaagtcccagtccccggagcagagagaagtggggccggcccggggcgctggtggtggctgtctggacacgtccttagcgcctgggaaccaggacataaaagcgcctccggagccgccctgcggcggggtccctttcatcccacttaaagtgcttctgcccctagggtttccggagggagagccgagatgggatgggggagcctgggggtcccccttggcaggggtgtctctttctggtttggagggttgttgctgtaaaaataactcctttgatgagcttccttattaaccctttcagacccagtctgttggagccatgaaggaagagggaaagagggctgccattcctgacagcctcccagccagggctggcgataaaggaccgagatagatggagggggcgagtagggaagtcctcttctaaaatgagagatagggatttggtggggtatggaaggaactaaccccttccctctccacctctgattcagcccttaattcttggtctatgataaataaagttcagtagtctcacattccccatctattaccctaggtgtgttttcaaggcagccagcggtagaatccatgtagttcccaccagttgccttcccctcagggatggaaggaagagggtttcttgggctggttgagggcagattgggggtgtctcatcagagggacctccactggttcccactcagagtggaggcctgcagcctacctgaccatctctttagctgtcaccaagaaaataaaccccactgtctctctagcttggcccttgtctttcccttgcccctgccatagcatgttcattaggggattccttcctccccctcatctcacaggggaagggagaggaaagagttgttctcccactggaaggggttctgccttctgaggtgacatccaggaagctgtccccattcccttctcctttagatgctagaaacacattttgattctgatcatggggtgggggagagaggaaaggagggaggggagaagcccagcagaagctgagccaggcagaggggaaagaagctgatatgaggaagggtctgacaggccacagcccttgcagccggagggctttcccacactcaagagaggggccttacagtccctctgacacccctcccccttcccctcgctccctttcttcacccggagccctctgcagagattagctgtgtattgatttttaagttataagcaaagggtattttatttaatattaggttatgtgtgtgcatgttgtgtgtacctgtgtgcatgtatgtgtgtttctctactgagcctggggtctctagccagggagaccccatcttattcaccatgtccaagatcctgggatctgggcccagcatctcttcctcctttgtagatgctggagcccagccaaggtctgggagctatatgggaagtgggggctgggatctgggtgggaatatgtgtttgtatacaaaggggccctccttaaaagggacaggatgaccttcccgaggaactcattggcctggggtagtttaagaagtaatgttctttctttctttctcttttccctacctcctgctaacccaaccagagatccccttccttgctgagagggttgggggcaggaggagatttggcagtgcctgcaggttgcctggccaggtggagagggggaaagaggaagggcaccgtgggtgtaagatgcctttctcctccacccatcgaaaccagccaccccttccctgtgccaccaagacagccttttccagtggccatcctaaggggaactcccaaatgggtgttgctggtggacacagatgctccccccaatggaagccccaagctctgaggtatgcgggtagaggctttggataggttttcttctgctcccctcttttatagatctaggctgcttggctgcctgtctttctaggcagtccccctagaggaaaaatgtaggaatttattttttctttaactgctgtgaactcactttgagggggtaggaggagggagaaacagcctgtgttttttatgcaataaagtcatcaactac

SEQ ID NO: 32 - DNA, 3681 bp, Homo sapiens
gtaattgctgcggggaggacaggccagctctggaagaaaacaggtggacctgggtcccattaacctggacagacacctccaagatgagcatgagggggctgctggatctgttctcctttcacagctgtatcctgaagaatggcagtgagccggaagagccacaggattcggcaaagacggacacacagaaatgggttctcactacgttgcccaggctggtctggaactcctagactcaagcgatcctcccacctcagcctcccaaagcactgggattacagacatgagccaccgcacccggtcaggtgcagccgttggggactgcaaagctgttgatgcagaaggcctagtgcctggggatgcccacattgctggtgtctgccacattcaggatgcctgaggtccgggagccaggaagggtgaagtagtccctctgtcggccgctgttgaagcctgcctgggggtgtcatttggaacagggtcagcccacctgcgtcctcaccctccccaaggacccagggaggcccctgtgggttccccactcacctgggccgcaatgccccccaggccgtggtgggggctcccaccgctggccacccccatggtccactggatgtccttgtggtgaaacagcacccaggaggggccgctgcccagggccagcacctcctggaaggtgttcacctgcagggcaagcgccaggagccagggtgtcgtgggctggtccaggggcacccaggaccctcctcactgcccagtcctcaaccgagctccatctgcaccactaagctggctgcttccagctgtgccacctcctgggcccccatccacaccacatcccgggcccctacccatgccacatcccagacccccatccacgccacctcccaggcccctgtccacaccatgtccacagctgcctctgctggcacctgtgccagccttcctttcacatcccccagtctcgatttttctgctctgctctcatcccctctctctctccgtctctctccttttcctattctctgtctctccatctgatcatctcactcctctcgcttgctgtcttgcctcctctctctcgccatttctctccctgttcgtgtcttccctctttctcacccattttctctacctcgctgcatctccatgcttccgtctctctgtctctctcttcccgccccctctatctctccctcgttcccccatctccgtctctcctccgtggtgtctcctccttacccaggaatccaagcccttcttcccaaggggttgggccaaacagcctcagcctgggcccttctctgccacccgcttcctcacctggggaccaagtgccccgtaaaatggaatttggtcccacatggccaccagcagggcgcaaggggtgggcggggcatgggggaaggctgaggccaggtctcaggccacctgctgcggcagctctggctggcggctctgccggtaccacatctggcccctgctcagtgtggacccattggcccagaacgggactgcgaaggcctggcccatctcctggggaaatgcctccagtgtgaagagagccacaggctccccgaaggacaccagcccattggtgcagaactgggggaagtgaggaagggcaagacccgcaggggggtgttggcagggcaggggacagaagagggacagggcccgccccaggaagacagaggacgaagccagaaggagccaggagctatagatatagacaggctccgagtcaagagtggggtggggagaagagagagagcccagggaccgcacggtcagggccagggcactcacataggccgtcccgtgtgtggcttcaaagagcatgaagggctccagcagccgcagctcctcagagaagtcatcatcctctgcagggagggcctgatccccacactccagcccataggggtacaggagcgaggctggggacacgagaccactgaggctcccctcagagccccagggcagggctgtgtcccctcagtccccaagagaaagctccaggtcccctgagggactccagggtggggccatggctcctcagacccctggagccctccctgcctgtccacatggccctgtcctctctccaggttagggagcacatccattctcacctctgatctccttgctccaagtcacttggttctgtaggactgaggggaaggatgtcagctaagggctggggcccacgtggcacagtcgacccccttccagagtccccactcctactcacccaccagcagaagccacagtatccccatggtggctgcaggtgctgcgggccaggcgggcttataccagggcagaggtggggccagggtggggacggggcagctggagctcaccttccattgttcgagggtccagggggcctgggggtctcccagggatcctggttcccatccggtctgcctgaggctgggccaggtctggggttggtgggcagggcaagggaggaaagaggggatggaaatcttccagggctttcccagggggccgggttgccagaccctggaggaaccccccacccattagcagggctgggcacaagtcaagcgatccacagtgggaaagttgagccactgcttggtgaagggccgctgctgacagacagctgaacatgcagggagcctcttcccatggggccctgctggttctcttggagcaggttagagatgagcacacagcatccaggaacggagtgcatgtgcatcaagcagggccaagatgtgtggctggggagactcgtgggctgctggccagccccgggggcccaggggtgggcatctgcagggcatggctggggctgccatggtggatagtcaaggtcaggcatttaggagtgttactggacccagaaggggagattcgcctggagacgtgaacggggagacggggaggaggagcatacaggcaagggggctcgttactgtgcacctgtgagattcacggaccaccctggtggaggaggctcagagttaggcactggggactccatcttcaaagcagtgtcccaaaggggtgctccagaccctcaaaccccagacagccctttacctggtcaaaccacatgggacagagggtcacctgtgttcctggaccaaactgaggattaggctgctatttctcatggcccagtgatgagatgcagataaactgggagaacagggaggttttttttgtttttgtttttgtttttgtttttgttttttgagacggagtctcgccctatcgcccaggctggagtgcaatggcactgtctcggctcacggcaacctctgcctcccgggttcaagcaattctcctgcctcagcctcccaagtagttgggattaccaacacccaccaccatgcctggctaatttttgtattattagtagagacggggtttctccatgttggtcaggcttgtctcgaattcctgacctcaggtgatccgcctacctcggcctcccaaagtgctgggattacaggcatgagccaccgcacccagcgaaaagggagtttttatttctgtaactggttatagggtgaaagcctggaaattgtccccagaccaactcaaaattacaaagttttccagagcttatataccttctaagctatatgcctgtgtgtaagtgtagtttcttcagacccccaattaaacttgtttaatcct

SEQ ID NO: 33 - DNA, 3523 bp, Homo sapiens
atggctgtgcgctctcgccgcccgtggatgagcgtggcattagggctggtgctgggcttcaccgccgcgtcctggctcatcgcccccagggtggcggagctgagcgagaggaagagacgtggctccagcctctgctcctactacggtcgctctgctgctggcccccgcgccggcgctcagcagccgctcccccagccccagtcccgaccacggcaggagcagtcgccgccccccgcgcgccaggatctccaggggccaccgctgcccgaggcagcacccgggatcaccagttttcgaagcagcccctggcagcagccacctccgctgcagcagcggcggcgaggacgcgagcctgagggcgcgacggggcttcccggtgctccagcggccgagggggagcccgaggaggaggacgggggcgcggctgggcagcggagagacggccggccggggagtagccacaacggcagcggggacgggggcgctgccgccccgagcgcccgaccccgggacttcctgtacgtgggggtgatgaccgcgcagaagtacctgggcagccgcgcgctggccgcgcagcggacctgggcgcgtttcatcccgggccgcgtggagttcttttccagccagcagccccccaacgccggccagcccccgccacccctgcctgtcatcgcgctaccgggtgtggacgactcctatcctccccagaaaaagtccttcatgatgatcaagtacatgcacgaccactacctggacaagtatgagtggttcatgcgcgccgacgacgatgtctacatcaaaggtgataaattagaagagtttcttagatcgctaaacagcagtaagcctctctacctgggacagactggcctggggaatattgaagagcttggaaagctgggactggagcctggggaaaacttctgtatgggaggacctggcatgatctttagccgagaagttctcaggaggatggtgccacatattggtgaatgccttagagaaatgtacacgactcatgaggatgtggaagtaggaagatgcgttcgccgttttggtgggactcagtgtgtctggtcttacgagatgcaacaactgttccatgaaaattatgaacacaatcggaagggttacatccaagaccttcacaatagcaaaatccatgcagccataacacttcatcccaacaaaaggcctgcataccaatacaggctgcataattacatgctcagccgcaaaatttctgaacttcgctaccgcaccatccagctccacagggaaagtgccctgatgagcaagctcagtaacacagaagtgagcaaagaggaccagcagctgggagtgataccttctttcaaccacttccagcctcgggagagaaatgaagtgatagaatgggagttcctgacagggaagcttctatactcagcagctgagaaccagccccctcgacagagcctcagtagcattttaagaacagcactggatgataccgtcctacaggtgatggagatgatcaatgagaatgccaagagcagaggacggctcattgacttcaaggaaattcagtatggctaccgcagagttaaccccatgcacggggtggagtacattttggatttactccttttatacaaaagacacaagggaaggaaactgactgtgccagtgagacgtcatgcctatcttcagcagttgttcagcaagcctttcttcagagagaccgaagagctagatgtcaacagtcttgtggagagtattaacagtgaaactcagtcattctcctttatatctaattctttaaagatattatcttcttttcaaggtgccaaagaaatgggagggcacaatgaaaagaaagtacacattctcgttcctctcatcggaaggtatgacattttcttgagattcatggagaactttgaaaacatgtgtcttatcccaaagcagaatgtaaagttggtcattatccttttcagtagggattctggccaagactccagcaagcatattgagctgataaaagggtaccagaacaagtaccccaaagcagaaatgaccctgatcccaatgaagggagagttttccagaggtcttggtcttgaaatggcttctgcccagtttgacaatgacactttgctgctattttgtgatgttgacttgatcttcagagaagattttctccaacgatgtagagacaatacaattcagggacaacaggtgtactatcccatcatctttagccagtatgacccaaaggtaacaaacgggggaaatcctcccactgatgattacttcatattctcaaaaaagactggattttggagagactatggatatggcatcacctgtatttacaaaagtgatcttctaggtgcaggtggatttgatacctcaatacaaggctggggactagaagatgtagatctctacaataaagtcattctatctggcttaaggccattcagaagccaagaagtaggagtggtgcatattttccatccagttcattgtgatcctaacttggaccctaagcagtataagatgtgcttaggatccaaggcaagtactttcgcctcaaccatgcaactggctgaactctggcttgaaaaacatttaggtgtcaggtacaatcgaactctctcctgacagtccaggcaacacattttgccttttttaaggggagtttacctcattgttggttgttgttatttttattgtattattgttattttattattattattgttataattttattttgttgtcctggtcttaaactactcttggttgtcttcctaagggtgtttgttgacctcaagcaagaagagtctgcagttcactgatgtttcagatttctactgaagtcaatatgttattacttttatataactttatttgagattgagttaaatcatagcaatcaaatgacttttgaaacgcattcatcacaagagacagcttagaagaatgttctcttggggcttaaagatgcaatatcgacttttatttggtttctcaaattcaatgtgatacaaatatttactggtgaaaggtaccacaaaagtgctttatgcttcctatggggaagggactctgtaacataaacttgagttttgtaatttatacaaggacttaaatatataaacaataattttcttcatctttagaatttttaaagtaaatgaatacctatgattgtatgtttattttttaaaaaaagttttaaaaattgaggagttttgttccacaagcaaccggtactggctaccctggtcttatgacaattatgagctcctcacaactgtctgctataaatgcgaatgaactttattttctcaaggaaatatgatttaattaaatattcatgtacattttagaagctttatgaaacaatgtccttcatttgctggcaagaagataaaatatgacagaacctgtttatttaaaataaaacacaggtatagcagtattctttttcaaaaacactgaaaaaaaaaaaaaaaaaaaaaaaaaaaaaaaa

(19) United States
(12) Patent Application Publication
(10) Pub. No.: US 2010/0075334 A1
(43) Pub. Date: Mar. 25, 2010

(54) METHYLATION BIOMARKER FOR EARLY DETECTION OF GASTRIC CANCER

(76) Inventors: Yong Sung Kim, Daejeon (KR); Seung Moo Noh, Daejeon (KR); Hyang Sook Yoo, Daejeon (KR); Jeong Hwan Kim, Daejeon (KR); Mi Rang Kim, Daejeon (KR); Hay Ran Jang, Daejeon (KR); Kyu Sang Song, Daejeon (KR); June Sik Cho, Daejeon (KR); Seon-Young Kim, Daejeon (KR); Hyun Yong Jeong, Daejeon (KR)

Correspondence Address: JHK LAW, P.O. BOX 1078, LA CANADA, CA, 91012-1078, US

(21) Appl. No.: 12/596,245

(22) PCT Filed: Apr. 15, 2008
(86) PCT No.: PCT/IB08/03482

§ 371 Date: Oct. 16, 2009

Related U.S. Application Data
(60) Provisional application No. 60/912,115, filed on Apr. 16, 2007.

(51) Int. Cl. C12Q 1/68

(52) U.S. Cl. 435/6

(57) ABSTRACT

The present application describes a method of diagnosing gastric cancer or a stage in the progression of the cancer in a subject comprising assaying for loss of expression of a marker gene such as POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, or CHSY3, or a combination thereof.

### CROSS-REFERENCE TO RELATED APPLICATIONS

The present application is a continuation-in-part application of PCT/IB2008/003482, filed Apr. 15, 2008, which claims benefit of priority to U.S. Provisional Application No. 60/912,115, filed Apr. 16, 2007, the contents of which are incorporated by reference herein in their entirety.

### BACKGROUND OF THE INVENTION

1. Field of the Invention

The invention relates to a systematic approach to discovering biomarkers in gastric cancer cell conversion. The invention relates to discovering gastric cancer biomarkers. The invention further relates to diagnosis and prognosis of gastric cancer using the biomarkers. The invention further relates to early detection or diagnosis of gastric cancer.

2. General Background and State of the Art

Over the past several years, many studies have demonstrated that multiple genetic or epigenetic alterations are responsible for the development and progression of gastric cancer (GC) (1 Zheng et al., 2004). Advances in diagnostic and treatment technologies have resulted in excellent long-term survival for GC, but it is still the second most common cause of death from cancer worldwide (2 Parkin et al., 2005). Recent studies showed that epigenetic alterations as well as genetic alterations occur throughout all stages of tumorigenesis including the early phases and that those epigenetic alterations occurred much more frequently than genetic alterations (3 Zardo et al., 2002). The silencing of tumor suppressor genes is increasingly recognized as a key driving force in the development of cancer involved (4 Jones & Baylin, 2002).

The detection of epigenetic alteration in tumorigenesis has led to a host of innovative diagnostic and therapeutic strategies. Epigenetic changes have been detected in the body fluids of almost every organ system in cancer patients (5 Laird, 2003). For many epigenetically silenced genes, re-expression in tumor cells can lead to suppression of cell growth or altered sensitivity to existing anticancer therapies and small molecules that reverse epigenetic inactivation are now undergoing clinical trials in cancer patients (6 Momparler et al., 1997, 7 Pohlmann et al., 2002). Thus, epigenetic alterations are not only potential therapeutic targets because of their reversibility, but also potential biomarkers that can be used to detect and diagnose cancer in its earliest stages (8 Brown et al., 2002).

GC is histologically classified into two subtypes, the intestinal and the diffuse types (9 Lauren, 1965). The precise mechanism underlying both types of gastric carcinogenesis is not fully understood. However, many reports regarding gene hypermethylation in gastric carcinogenesis have been published recently by the gene-by-gene approach. For example, APC which is mutated in 25% of intestinal-type GC (10 Tamura et al., 1994), is frequently hypermethylated in sequential carcinogenic steps, even in normal gastric mucosae (11 Clement et al., 2004). CDH1, of which loss of function by mutation is pivotal in both sporadic and hereditary forms of diffuse-type GC (12 Becker et al., 1994), is hypermethylated more frequently in diffuse-type GCs than in intestinal-type GCs (13 Oue et al., 2003). Three tumor suppressor genes, p15/INK4B, p16/INK4A and p14/ARF, are frequently hypermethylated in GC, but there is a contrast in methylation patterns between these genes. The methylation prevalence of p15/INK4B and p16/INK4A is relatively tumor-specific, whereas p14/ARF hypermethylation occurs in precursor lesions as frequently as in tumors. Also, p16/INK4A is hypermethylated largely in intestinal-type GC (14 Iida et al., 2000), whereas p14/ARF methylation occurs predominantly in diffuse-type GC (13 Oue et al., 2003). RUNX3, in which the main cause of loss of function is hypermethylation (15 Li et al., 2002), could play a role in cell differentiation and proliferation in intestinal-type gastric carcinogenesis.

Toyota et al. proposed a novel molecular phenotype based on gene hypermethylation in cancer (16 Toyota et al., 1999a). They identified 26 hypermethylated CpG islands (called “MINT”: methylated in tumor) in colorectal cancers and classified the MINTs into two types: age-related methylated genes (type-A) and cancer-specific methylated genes (type-C). They also found a frequent hypermethylation of type-C MINTs in a subset of cancer, and designated this phenotype the CpG island methylator phenotype (CIMP). This CIMP+ phenotype was also discovered in 24-47% of GCs (13 Oue et al., 2003, 17 Toyota et al., 1999b). The presence of CIMP indicates that multiple promoter regions of genes are methylated in many gastric cancers. Moreover, CIMP has been detected not only in tumors but also in premalignant lesions (18 Lee et al., 2004). This finding suggested that CIMP could represent one of the early molecular events in gastric carcinogenesis. Although we know many of gene hypermethylation in gastric carcinogenesis, the gene set should be limited to understand the overall methylation level of gastric carcinogenesis and to diagnose cancer in its earliest stages.

In this study, we analyzed primary gastric carcinomas as well as gastric cancer cell lines to identify novel epigenetic targets by using Restriction Landmark Genomic Scanning (RLGS) analysis (19 Hatada et al., 1991), which was successfully applied to address global methylation in various human carcinomas (3 Zardo et al., 2002, 20 Nagai et al., 1994, 21 Costello et al., 2000, 22 Rush et al., 2004). To our knowledge, no genome-wide analysis of CpG island methylation has been reported in GCs. We also examined the expression mode of epigenetic targets in a set of clinical samples and its correlation between the targets or between the targets and CDH1 or DAPK, well-known tumor suppressor genes frequently silenced in GC. In addition, we first report TCF4, which encodes transcription factor 4 as one of basic helix-turn-helix transcription factor, to be an age-related methylated gene (type-A) as well as a cancer-specific methylated gene (type-C) in GC by the quantitative methylation analysis.

Accordingly, the present invention is directed to screening for methylated markers involved in cell conversion, especially cancer cell conversion and treatment of cancer.

### SUMMARY OF THE INVENTION

Epigenetic alterations are now recognized to be common features of human solid tumors, though global DNA methylation has been difficult to assess. We report the first measurement of global methylation in gastric cancer and find 3.3% NotI sites methylated in 15 gastric tumors and 11.9% NotI sites methylated in 11 gastric cancer cell lines using Restriction Landmark Genomic Scanning (RLGS). By mixing RLGS gels with NotI-linked clones, we identified 26 epigenetic targets, which were frequently methylated in RLGS profile and correlated with gene silencing by RT-PCR. We confirmed that 23 of 26 genes were restored by demethylating agent 5-aza and/or HDAC inhibitor TSA. Twenty-three genes showed loss of expression in range of 25˜50% in 96 primary tumors compared to normal counterpart by quantitative real-time PCR analysis. In particular, we found combined expression of LIMS2, ALOX5, TCF4, PRKD1, and NACM2 in the tumor samples. Of those, the loss of expression of TCF4 was significantly high in early gastric cancer type (P=0.004) or early stage (P=0.013) and in intestinal type (P=0.0001). By using pyrosequencing analysis, we quantitated the methylation status in exon 1 of TCF4 and found strong correlation between gene silencing and hypermethylation on exon 1 in primary tumors as well as cell lines. We also found that the methylation status in primary tumors was highly correlated with patient's age (R=0.3265, P=0.0037, 34.7% mean methylation). Furthermore, the methylation even in normal tissue showed high correlation with patient's age (R=0.4524, P<0.0001, 13.2% mean methylation), indicating that the methylation was dramatically increased from 50 years in normal mucosa tissue. Therefore, our results suggest that in a preferred embodiment, the methylated TCF4 has the potential to be an early-detection and prognostic biomarker for gastric cancer, which is also useful for monitoring cancer by assaying for the methylated TCF4.

The present invention is based on the finding that by using the system described in the present application, several genes are identified as being differentially methylated in gastric cancer as well as at various dysplasic stages of the tissue in the progression to gastric cancer. This discovery is useful for gastric cancer screening, risk-assessment, prognosis, disease identification, disease staging and identification of therapeutic targets. The identification of genes that are methylated in gastric cancer and its various stages of lesion allows for the development of accurate and effective early diagnostic assays, methylation profiling using multiple genes, and identification of new targets for therapeutic intervention. Further, the methylation data may be combined with other non-methylation related biomarker detection methods to obtain a more accurate diagnostic system for gastric cancer.

In one embodiment, the invention provides a method of diagnosing various stages or grades of gastric cancer progression comprising determining the state of methylation of one or more nucleic acid biomarkers isolated from the subject as described above. The state of methylation of one or more nucleic acids compared with the state of methylation of one or more nucleic acids from a subject not having the cellular proliferative disorder of gastric tissue is indicative of a certain stage of gastric disorder in the subject. In one aspect of this embodiment, the state of methylation is hypermethylation.

In one aspect of the invention, nucleic acids are methylated in the regulatory regions. In another aspect, since methylation begins from the outer boundaries of the regulatory region working inward, detecting methylation at the outer boundaries of the regulatory region allows for early detection of the gene involved in cell conversion.

In one aspect, the invention provides a method of diagnosing a cellular proliferative disorder of gastric tissue in a subject by detecting the state of methylation of one or more of the following exemplified nucleic acids: POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, CHSY3, or a combination thereof.

Another embodiment of the invention provides a method of determining a predisposition to a cellular proliferative disorder of gastric tissue in a subject. The method includes determining the state of methylation of one or more nucleic acids isolated from the subject, wherein the state of methylation of one or more nucleic acids compared with the state of methylation of the nucleic acid from a subject not having a predisposition to the cellular proliferative disorder of gastric tissue is indicative of a cell proliferative disorder of gastric tissue in the subject. Some of the exemplified nucleic acids can be nucleic acids encoding POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, CHSY3, or a combination thereof.

In yet another embodiment, the invention is directed to early detection of the probable likelihood of formation of gastric cancer. According to an embodiment of the instant invention, when a clinically or morphologically normal appearing tissue contains methylated genes that are known to be methylated in cancerous tissue, this is an indication that the normal appearing tissue is progressing to cancerous form. Thus, a positive detection of methylation of gastric cancer specific genes as described in the instant application in normal appearing gastric tissue constitutes early detection of gastric cancer.

Still another embodiment of the invention provides a method for detecting a cellular proliferative disorder of gastric tissue in a subject. The method includes contacting a specimen containing at least one nucleic acid from the subject with an agent that provides a determination of the methylation state of at least one nucleic acid. The method further includes identifying the methylation states of at least one region of at least one nucleic acid, wherein the methylation state of the nucleic acid is different from the methylation state of the same region of nucleic acid in a subject not having the cellular proliferative disorder of gastric tissue.

Yet a further embodiment of the invention provides a kit useful for the detection of a cellular proliferative disorder in a subject comprising carrier means compartmentalized to receive a sample therein; and one or more containers comprising a first container containing a reagent that sensitively cleaves unmethylated nucleic acid and a second container containing target-specific primers for amplification of the biomarker.

In one embodiment, the invention is directed to a method of identifying a converted gastric cancer cell comprising assaying for the methylation of the marker gene.

In yet another embodiment, the invention is directed to a method of diagnosing gastric cancer or a stage in the progression of the cancer in a subject comprising assaying for the methylation of the marker gene.

In another embodiment, the invention is directed to a method of diagnosing likelihood of developing gastric cancer comprising assaying for methylation of a gastric cancer specific marker gene in normal appearing bodily sample. The bodily sample may be solid or liquid tissue, serum or plasma.

In yet another embodiment, the invention is directed to a method of assessing the likelihood of developing gastric cancer by reviewing a panel of gastric-cancer specific methylated genes for their level of methylation and assigning level of likelihood of developing gastric cancer.

In one aspect, the invention is directed to a method of diagnosing gastric cancer or a stage in the progression of the cancer in a subject comprising assaying for loss of expression of a marker gene, which is selected from the group consisting of: POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, and CHSY3, or a combination thereof. The loss of expression may be caused by hypermethylation of the marker gene. The hypermethylation may occur in a regulatory region or an amino acid encoding region. The stage referred to may be early TNM (Tumor, Node, Metastasis) stage, and optionally the TNM stage may be stage I. Preferably, the marker gene may be TCF4, PRKD1, CYP1B1, LIMS2, ALOX5, or BACH2, or a combination thereof. In one embodiment, the marker gene may be TCF4, or preferably, the methylation of TCF4 may occur in exon I.

Further in the method described above, the gastric cancer may be intestinal type. Preferably, the marker gene may be TCF4, PRKD1, CYP1B1, LIMS2, ALOX5, or BACH2, or a combination thereof. Preferably, the marker gene may be TCF4, and methylation of TCF4 may occur in exon I.

In another aspect, the invention is directed to a method of diagnosing likelihood of developing gastric cancer comprising assaying for methylation of a gastric cancer specific marker gene in normal appearing bodily sample. The bodily sample may be solid tissue, or body fluid. Preferably, the marker gene may be TCF4, PRKD1, CYP1B1, LIMS2, ALOX5, or BACH2, or a combination thereof.

In another aspect, the invention is directed to a kit that includes

(i) a carrier means compartmentalized to receive a sample therein, and

(ii) one or more containers comprising a first container containing a reagent which sensitively cleaves unmethylated cytosine, a second container containing primers for amplification of a CpG-containing nucleic acid, and a third container containing a means to detect the presence of cleaved or uncleaved nucleic acid.

Preferably, the nucleic acid may be a marker gene for detection of gastric cancer. In this kit, the marker gene may be POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, or CHSY3, or a combination thereof. The nucleic acid in the kit may be a marker gene for detection of early gastric cancer. In particular, the marker gene may be TCF4, PRKD1, CYP1B1, LIMS2, ALOX5, or BACH2, or a combination thereof.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1B show RLGS profile using NotI-EcoRV-HinfI restriction enzymes in gastric cancer. (A) A standard RLGS profile from normal mucosa DNA displaying nearly 2,300 NotI fragments. For the comparisons of RLGS profiles from different samples, each spot is given a three-variable designation (Y coordinate, X coordinate, spot number). The central region of the RLGS profile used for all comparisons in this report has 30 sections (1-8 vertically and A-D horizontally), containing about 1,948 spots by our previous work (25 Kim et al., 2006). (B) The representative examples for comparison between RLGS profiles in an enlarged view. Each arrowhead indicates RLGS spot in normal tissue but with decreased intensity in its tumor relative to that in the normal. The spots were completely absent or decreased in the corresponding gastric cancer cells (Cell), and were seen at about half intensity (Cell+Normal), when cells and normal DNA were mixed to confirm the position of the spots.

FIGS. 2A-2C show gene selection with NotI-methylation in gastric cancer cell. (A) Variability of gene expression across 11 gastric cancer cell lines by RT-PCR analysis. The genes or mRNAs selected in this study are shown as symbol or accession number on the left. Gastric cancer cell lines are shown at the top of the respective lanes. (B) Reactivation analysis after drug treatment. This analysis was done with three gastric cancer cell lines, SNU001, SNU601, and SNU638. 5-AZA and TSA are abbreviations of 5-aza-2′deoxycytidine and trichostatin A. (C) Correlation between ‘loss of expression’ (LOE) and NotI-methylation in primary tumors. Genes are arranged from left to right with the highest degree of LOE in the last column of Table 1. For the comparison, % of NotI-methylation was arbitrarily calculated from 3rd column of Table 1 and arranged by the side of LOE for each gene. Open and closed quadrangles indicate % of LOE and % of NotI-methylation by RLGS, respectively. The degree of LOE and NotI-methylation was plotted for each gene, showing a highly positive correlation between two values by linear regression analysis.

FIGS. 3A-3C show correlation of gene expression between selected genes and comparison of CDH1 and TCF4 expression in gastric carcinogenesis. (A) Strong correlation of PRKD1, CYP1B1, LIMS2, ALOX5, and BACH2 with TCF4 was shown at the top. Middle figure showed a strong correlation between CDH1 and DAPK, but the two genes had no correlation with TCF4. No correlation of PRKD1, CYP1B1, LIMS2, ALOX5, and BACH2 with CDH1 was also shown on the bottom. These figures were drawn from Table 2 data. (B) Comparison of CDH1 expression in 96 paired samples. Quantitation was achieved by real-time RT-PCR and each expression value normalized by GAPD expression from each tumor was divided by the normalized value from normal tissue. The boxes indicate the 25^{th} through 75^{th} percentiles and the whiskers indicate the 90^{th} and 10^{th} percentiles. Mean expression value was compared between each specimen group by Student's t-test: early (EGC) and advanced gastric cancer (AGC); four TNM stages (I, II, III, and IV); intestinal (I) and diffuse type (D). Numbers in parentheses below each tumor type indicate the number of samples examined. (C) Comparison of TCF4 expression in 96 paired samples. Plotting and statistical comparison between specimen group were followed in the same procedure as the above.

FIGS. 4A-4G show methylation analysis at TCF4 exon 1. (A) Strategy for methylation analysis based on gene structure of TCF4. According to UCSC Genome Bioinformatics database, TCF4 gene consistes of 19 exons ranging of 360 kb on 18p11.21 of human chromosome and a typical CpG island (CpG30) is found at 1.5 kb apart from transcription start site. NotI sequence (6B54) cloned in this study is located in intron 7 and another CpG cluster can be found at 5′-upstream region encompassing the exon 1. (B) Methylation-specific PCR was performed at NotI site in the intron 7 and CpG cluster region at the exon 1 for 11 gastric cancer cell lines. The result was compared with TCF4 expression by RT-PCR. Gastric cancer cell lines are shown at the top of the respective lanes. N indicates normal tissue. (C) Pyrosequencing results at TCF4 exon 1. Based on TCF4 exon 1 sequence shown in FIG. 4A, quantitative methylation status at seven CpG sites was analyzed for 10 gastric cancer cell lines by pyrosequencing assay. Each cell line was shown on the left and mean % of methylation at seven CpG sites on the right. (D) Pairwise comparison of methylation status in 85 paired normal and tumor DNAs. Mean % of methylation was 13.2% but 34.7% in their tumor DNA, showing a highly significant difference (Student's t-test, P>0.0001). (E) Correlation of TCF4 expression with TCF4 exon 1 methylation. For comparison, relative methylation for each paired sample was arbitrarily defined as the degree of methylation in tumor minus that in normal DNA and plotted against relative expression by real-time RT-PCR, showing a negative correlation. (F) Comparison of TCF4 exon 1 methylation in 85 paired samples. The boxes and whiskers indicate mean % of methylation and standard deviation in each specimen group. No significant difference was found between EGC and AGC groups or between intestinal (I) and diffuse type (D). But a gradual change in methylation was found in normal and tumor DNAs along with patient age group. (G) Correlation of TCF4 exon 1 methylation with aging. Regression result of methylation with aging was shown on the left for normal DNAs and on the right for tumor DNAs. Both showed a highly significant correlation.

FIGS. 5A-5K show methylation analysis of various genes including (A) CDH1, (B) DAPK, (C) ALOX5, (D) BACH2, (E) CYP1B1, (F) LIMS2, (G) PRKD1, (H) TCF4, (I) POPDC3, (J) CCDC67, and (K) LRRC3B-Graphs under Pairwise column show pairwise comparison of methylation status in paired normal and tumor DNAs. Graphs under Normals and Tumors columns show correlation and regression results of methylation with aging for normal DNAs and tumor DNAs, respectively. Graphs under Correlation show correlation of gene expression with methylation. For comparison, relative methylation for each paired sample was arbitrarily defined as the degree of methylation in tumor minus that in normal DNA and plotted against relative expression by real-time RT-PCR, showing a negative correlation.

### DETAILED DESCRIPTION OF THE PREFERRED EMBODIMENTS

In the present application, “a” and “an” are used to refer to both single and a plurality of objects.

As used herein, “cell conversion” refers to the change in characteristics of a cell from one form to another such as from normal to abnormal, non-tumorous to tumorous, undifferentiated to differentiated, stem cell to non-stem cell. Further, the conversion may be recognized by morphology of the cell, phenotype of the cell, biochemical characteristics and so on. There are many examples, but the present application focuses on the presence of abnormal and cancerous cells in the gastric tissue. Markers for such tissue conversion are within the purview of gastric cancer cell conversion.

As used herein, “demethylating agent” refers to any agent, including but not limited to chemical or enzyme, that either removes a methyl group from the nucleic acid or prevents methylation from occurring. Examples of such demethylating agents include without limitation nucleotide analogs such as 5-azacytidine, 5 aza 2′-deoxycytidine (DAC), arabinofuranosyl-5-azacytosine, 5-fluoro-2′-deoxycytidine, pyrimidone, trifluoromethyldeoxycytidine, pseudoisocytidine, dihydro-5-azacytidine, AdoMet/AdoHcy analogs as competitive inhibitors such as AdoHcy, sinefungin and analogs, 5′deoxy-5′-S-isobutyladenosine (SIBA), 5′-methylthio-5′deoxyadenosine (MTA), drugs influencing the level of AdoMet such as ethionine analogs, methionine, L-cis-AMB, cycloleucine, antifolates, methotrexate, drugs influencing the level of AdoHcy, dc-AdoMet and MTA such as inhibitors of AdoHcy hydrolase, 3-deaza-adenosine, neplanocin A, 3-deazaneplanocin, 4′-thioadenosine, 3-deaza-aristeromycin, inhibitors of ornithine decarboxylase, α-difluoromethylornithine (DFMO), inhibitors of spermine and spermidine synthetase, S-methyl-5′-methylthioadenosine (MMTA), L-cis-AMB, AdoDATO, MGBG, inhibitors of methylthioadenosine phosphorylase, difluoromethylthioadenosine (DFMTA), other inhibitors such as methinin, spermine/spermidine, sodium butyrate, procainamide, hydralazine, dimethylsulfoxide, free radical DNA adducts, UV-light, 8-hydroxy guanine, N-methyl-N-nitrosourea, novobiocine, phenobarbital, benzo[a]pyrene, ethylmethansulfonate, ethylnitrosourea, N-ethyl-N′-nitro-N-nitrosoguanidine, 9-aminoacridine, nitrogen mustard, N-methyl-N′-nitro-N-nitrosoguanidine, diethylnitrosamine, chlordane, N-acetoxy-N-2-acetylaminofluorene, aflatoxin B1, nalidixic acid, N-2-fluorenylacetamine, 3-methyl-4′-(dimethylamino)azobenzene, 1,3-bis(2-chlorethyl)-1-nitrosourea, cyclophosphamide, 6-mercaptopurine, 4-nitroquinoline-1-oxide, N-nitrosodiethylamine, hexamethylenebisacetamide, retinoic acid, retinoic acid with cAMP, aromatic hydrocarbon carcinogens, dibutyryl cAMP, or antisense mRNA to the methyltransferase (Zingg et al., Carcinogenesis, 18:5, pp. 869-882, 1997). The content of this reference is incorporated by reference in its entirety especially with regard to the discussion of methylation of the genome and inhibitors thereof.

As used herein, “early detection” of cancer refers to the discovery of a potential for cancer prior to metastasis, and preferably before morphological change in the subject tissue or cells is observed. Further, “early detection” of cell conversion refers to the high probability of a cell to undergo transformation in its early stages before the cell is morphologically designated as being transformed.

As used herein, “hypermethylation” refers to the methylation of a CpG island.

As used herein, a “methylation sensitive restriction endonuclease” is a restriction endonuclease that includes CG as part of its recognition site and has altered activity when the C is methylated as compared to when the C is not methylated. Preferably, the methylation sensitive restriction endonuclease has inhibited activity when the C is methylated (e.g., Sma1). Specific non-limiting examples of methylation sensitive restriction endonucleases include Sma I, BssHII, or HpaII, BstUI, and NotI. Such enzymes can be used alone or in combination. Other methylation sensitive restriction endonucleases will be known to those of skill in the art and include, but are not limited to SacII, and EagI, for example. An “isoschizomer” of a methylation sensitive restriction endonuclease is a restriction endonuclease that recognizes the same recognition site as a methylation sensitive restriction endonuclease but cleaves both methylated and unmethylated CGs, such as for example, MspI. Those of skill in the art can readily determine appropriate conditions for a restriction endonuclease to cleave a nucleic acid (see Sambrook et al., Molecular Cloning: A Laboratory Manual, Cold Spring Harbor Press, 1989).

As used herein, “predisposition” refers to an increased likelihood that an individual will have a disorder. Although a subject with a predisposition does not yet have the disorder, there exists an increased propensity to the disease.

As used herein, “sample” or “bodily sample” is referred to in its broadest sense, and includes any biological sample obtained from an individual, body fluid, cell line, tissue culture, depending on the type of assay that is to be performed. As indicated, biological samples include body fluids, such as semen, lymph, sera, plasma, and so on. Methods for obtaining tissue biopsies and body fluids from mammals are well known in the art. A tissue biopsy of stomach is a preferred source.

As used herein, “tumor-adjacent tissue” or “paired tumor-adjacent tissues” refers to clinically and morphologically designated normal appearing tissue adjacent to the cancerous tissue region.

Gastric Cancer Biomarkers

Although mechanistic insights into the molecular pathology of sporadic gastric cancers are increasing, the question of how carcinogenesis is initiated in human gastric mucosa tissues remains largely unanswered (1 Zheng et. al., 2004). However, it is widely accepted that epigenetic alterations are a prerequisite of virtually all tumors and that this epigenetic alteration facilitates the accumulation of further genetic alterations that result in cancer progression through clonal expansion of cells with a proliferative advantage (31 Grady, 2005).

RLGS technique (19 Hatada 1991) was used in this study to identify novel targets of promoter hypermethylation in a gastric cancer and to demonstrate the promoter hypermethylation in normal-appearing normal mucosae as well as the corresponding tumors. We found 3.3% of methylation on average by comparison of 1948 NotI-loci from RLGS profiles of 15 paired normal and tumor DNAs. Gastric cancer cell lines showed mean 11.9% of methylation, showing over 3-fold increase in global methylation in cell lines compared to primary tumors. The data is in good agreement with the previous observation that cancer cell lines exhibit significantly higher levels of CpG island hypermethylation than the primary malignancies they represent (26 Smiraglia et al., 2001; 32 Paz et al., 2003), suggesting that the majority of hypermethylation events in cell lines can be originated from background events, such as growth in culture. In fact, of the loci methylated in cell lines 72% were never methylated in 15 primary tumors examined. Although the CpG island hypermethylation found in cell lines was significantly different from that seen in the primary tumor they represent, nevertheless, the cell lines have retained some hypermethylation characteristics from their tumor of origin, particularly with regard to which loci become hypermethylated (26 Smiraglia et al., 2001). Thus, to identify novel targets of promoter hypermethylation in gastric cancer it is important to select loci methylated coincidentally in gastric cancer cells and primary tumors.

We had identified 40 NotI-loci, which were methylated coincidentally at least in two gastric cancer cells and two primary tumors and of which sequence information was available from previous literatures (21 Costello et al., 2000; 22 Rush et al., 2004; 25 Kim et al., 2006; 26 Smiraglia et al., 2001; 27 Rush et al., 2001; 28 Dai et al., 2001). Of those, a half has been described to be methylated in various tumor types in the previous publications: for example, the methylation of 3B79 NotI-locus, which corresponds to 3C18 of Master RLGS profile (21 Costello et al., 2000), has been detected in hepatocellular carcinoma previously (20 Nagai): the methylation of 3C43 NotI-locus (2D74 in Master RLGS profile) has been also detected in various tumor types such as breast carcinoma, colon carcinoma, and glioma (21), acute myeloid leukemia (27 Rush et al., 2001), and chronic lymphocytic leukemia (22). However, of genes or mRNAs linked to 40 NotI-loci, only 55% (22 of 40 transcripts) showed expression variability coinciding with NotI-methylation of RLGS data in gastric cancer cell lines. The remaining genes showed full-expression in all cell lines, no PCR product or no correlation with NotI-methylation, suggesting that their expression may be independent of NotI-methylation or not functional in gastric mucosa. Another possibility may be due to either misreading of the NotI-loci methylation or incorrect sequence information. Furthermore, after 5-Aza-dC and/or TSA treatment the above 22 genes were restored in the corresponding inactivated cells depending on gene-specific or cell-specific mode. Each target also showed a strong positive correlation between “loss of expression” (LOE) level and NotI-methylation for in primary tumors. Hence, the data indicates that the aberrant methylation event in selected genes has an impact on transcription in gastric cancer and may be associated with gastric carcinogenesis to a certain extent.

To our knowledge, the above 22 genes have never been reported in regard to gastric carcinogenesis. Of those genes, TCF4, SP6, EMX1, and BACH2 can be classified as transcription factors involved in the regulation of transcription, and POPDC3, KCNK9, NCAM2, DCBLD2, ADCY8, PRKD1, CHSY3, and LIMS2 as transmembrane proteins involved in cell growth or signal transduction based on the (Cancer Genome Anatomy Project) CGAP Web site at NCBI database or Human Protein Reference Database at Johns Hopkins University and the Institute of Bioinformatics. Also CAMK2N2, AMPD3, ALOX5, CYP1B1, CXXC4, and NOG are known to be implicated in cell growth through signaling transduction or metabolism. The remaining four genes are mRNA sequence or hypothetical protein with unknown function. Thus, the large portion of the selected genes has a wide variety of functions related with cell growth or signal transduction and may also be important in tumorigenesis for various cancer types including gastric cancer.

In the clinical samples tested, a significant association was found between CDH1 and DAPK expression, which were often reduced in various tumors by aberrant methylation in previous study (33 Esteller et al., 2001). The present data showed that the reduced expression of CDH1 was significantly associated with tumor depth or invasion. It is in good agreement with the previous observation that the LOE of CDH1 in gastric cancer was closely correlated with tumor invasion or micro-lymph node metastasis (34 Cai et al., 2001). However, CDH1 or DAPK showed no any other correlation with the selected genes in this study. Instead, we found a significant association between the selected gene expression and specifically a highly significant correlation between TCF4 and PRKD1, CYP1B1, LIMS2, ALOX5, or BACH2 expression. In particular, in contrast to figure of CDH1 the LOE of TCF4 was significantly high in early gastric cancer or early TNM (Tumor, Node, Metastasis) stage. In addition, TCF4 expression was significantly reduced in intestinal type rather than diffuse type. This picture can also be found for the other selected genes (data not shown). Therefore, the data suggests that the aberrant methylation of the selected genes including TCF4 may be associated with cancer initiation or early tumorigenesis rather than tumor invasion or metastasis during the gastric carcinogenesis.

The human TCF4 gene encodes transcription factor 4, a basic helix-turn-helix transcription factor. The protein at first has been known as ITF2 for ‘immunoglobulin transcription factor 2’ that binds to the mu-E5 motif of the immunoglobulin heavy chain enhancer and to the kappa-E2 motif found in the light chain enhancer (35 Henthorn et al., 1990) or as SEF2 for ‘SL3-3 enhancer factors 2’ that bind to a motif of the glucocorticoid response element (GRE) in the enhancer of the murine leukemia virus SL3-3 (36 Corneliussen et al., 1991). Recently it has been reported that TCF4 functions in concert with other TCF (T cell factor) target genes to promote growth and/or survival of cancer cells with defects in β-catenin regulation as a downstream target of the Wnt/TCF pathway (37 Kolligs et al., 2002), thus showing oncogenic property of TCF4. This implication is rather unexpected, because the present data had shown that the TCF4 expression was significantly reduced in association with NotI-methylation in our tissue samples examined. When the methylation status on TCF4 exon 1 was quantitatively examined in the clinical samples, a positive methylation was observed in many normal mucosae. This methylation may be due to ‘cancer cell contamination’ or ‘field cancerization effect’ (38 Slaughter et al., 1953; 39 Braakhuis et al., 2003). Nevertheless, we found a highly significant change in overall methylation status in primary tumors (34.7%) compared to that of their normal-appearing gastric tissues (13.2%), indicating that TCF4 exon 1 should be methylated in cancer-specific mode and so can be classified as type-C (16 Toyota et al., 1999a). Furthermore, we also confirmed a significant correlation between hypermethylation on TCF4 exon 1 and its reduced expression. No significant difference was found in the mean methylation levels between clinicopathologic parameters for tumor depth or Lauren's classification, though early or diffuse type in each parameter showed slightly high level.

Surprisingly, no methylation was detected on TCF4 exon 1 in any normal-appearing gastric tissues from 14 patients before age 50 year and the methylation status was gradually increased in dependent on aging after age 50 year. The data appeared to have great relevance to neoplasia since the incidence of sporadic gastric tumors is strongly age-related and increases logarithmically after age 50 years. This data is in agreement with a previous report demonstrating that hypermethylation of the ER-α promoter is apparent in colon carcinomas, including the earliest stages of tumor formation such as adenomatous polyps and even normal colonic mucosa with age-related mode (40 Issa et al., 1994). Thus, our result strongly suggests that the positive methylation in normal-appearing gastric mucosa can be due to ‘field cancerization effect’ rather than ‘tumor contamination’. This may be a first evidence for field cancerization in gastric mucosa, although the field cancerization has been described in many organ systems such as oral cavity, oropharynx, and larynx (102), lung (103), esophagus (104), vulva (105), cervix (106), colon (107), breast (108), bladder (109), and skin (110). Thus, TCF4 gene methylation may represent one of the earliest events that predispose to gastric cancer. We can define additionally TCF4 exon 1 methylation as type-A (16 Toyota et al., 1999a), because the methylation is significantly increased and dependent on aging not only in tumor tissues tissues but also in normal appearing tissues. In particular, it is interesting that the methylation status in normal-appearing gastric tissues after age 70 year is very similar to that in tumor tissues in age group less than age 50 year.

In conclusion, we identified 22 novel epigenetic target including TCF4 through the RLGS analysis. The data for TCF4 support the carcinogenesis model in which the development of a field with genetically or epigenetically altered cells plays a central role (39 Braakhuis et al., 2003; 31 Grady, 2005). In the initiation phase, that is, a normal gastric mucosa cell acquires epigenetic alterations and forms a “patch,” a clonal unit of altered daughter cells. These patches can be recognized on the basis of TCF4 exon 1 methylation. The conversion of a patch into an expanding field is the next logical and critical step in epithelial carcinogenesis. Additional genetic or epigenetic alterations are required for this step, and by virtue of its growth advantage, a proliferating field gradually displaces the normal mucosa. An important clinical implication is that fields often remain after surgery of the primary tumor and may lead to new cancers, designated presently by clinicians as “a second primary tumor” or “local recurrence,” depending on the exact site and time interval. Although the mechanism underlying the cause of these epigenetic alterations remains to be elucidated, these methylated genes have the potential to be early-detection and prognostic biomarkers for gastric cancer. Also detection and monitoring of field may have profound implications for cancer prevention.

Another embodiment of the invention provides a method for diagnosing a cellular proliferative disorder of gastric tissue in a subject comprising contacting a nucleic acid-containing specimen from the subject with an agent that provides a determination of the methylation state of nucleic acids in the specimen, and identifying the methylation state of at least one region of at least one nucleic acid, wherein the methylation state of at least one region of at least one nucleic acid that is different from the methylation state of the same region of the same nucleic acid in a subject not having the cellular proliferative disorder is indicative of a cellular proliferative disorder of gastric tissue in the subject.

The inventive method includes determining the state of methylation of one or more nucleic acids isolated from the subject. The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The nucleic acid of interest can be any nucleic acid where it is desirable to detect the presence of a differentially methylated CpG island. The CpG island is a CpG rich region of a nucleic acid sequence.

Methylation

Any nucleic acid sample, in purified or nonpurified form, can be utilized in accordance with the present invention, provided it contains or is suspected of containing, a nucleic acid sequence containing a target locus (e.g., CpG-containing nucleic acid). One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G*C base pairs. In certain regions, the density of CpG doublets reaches the predicted value; it is increased by ten fold relative to the rest of the genome. CpG islands have an average G*C content of about 60%, compared with the 40% average in bulk DNA. The islands take the form of stretches of DNA typically about one to two kilobases long. There are about 45,000 such islands in the human genome.

In many genes, the CpG islands begin just upstream of a promoter and extend downstream into the transcribed region. Methylation of a CpG island at a promoter usually prevents expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

In general, the CpG-containing nucleic acid is DNA. However, invention methods may employ, for example, samples that contain DNA, or DNA and RNA, including messenger RNA, wherein DNA or RNA may be single stranded or double stranded, or a DNA-RNA hybrid may be included in the sample. A mixture of nucleic acids may also be employed. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. The nucleic acid-containing sample used for determination of the state of methylation of nucleic acids contained in the sample or detection of methylated CpG islands may be extracted by a variety of techniques such as that described by Sambrook, et al. (Molecular Cloning: A Laboratory Manual, Cold Spring Harbor, N.Y., 1989; incorporated in its entirety herein by reference).

A nucleic acid can contain a regulatory region which is a region of DNA that encodes information that directs or controls transcription of the nucleic acid. Regulatory regions include at least one promoter. A “promoter” is a minimal sequence sufficient to direct transcription, to render promoter-dependent gene expression controllable for cell-type specific, tissue-specific, or inducible by external signals or agents. Promoters may be located in the 5′ or 3′ regions of the gene. Promoter regions, in whole or in part, of a number of nucleic acids can be examined for sites of CG-island methylation. Moreover, it is generally recognized that methylation of the target gene promoter proceeds naturally from the outer boundary inward. Therefore, early stage of cell conversion can be detected by assaying for methylation in these outer areas of the promoter region as well as in the amino acid encoding area of the gene, in particular the exon region.

Nucleic acids isolated from a subject are obtained in a biological specimen from the subject. If it is desired to detect gastric cancer or stages of gastric cancer progression, the nucleic acid may be isolated from gastric tissue by scraping or taking a biopsy. These specimens may be obtained by various medical procedures known to those of skill in the art.

In one aspect of the invention, the state of methylation in nucleic acids of the sample obtained from a subject is hypermethylation compared with the same regions of the nucleic acid in a subject not having the cellular proliferative disorder of gastric tissue. Hypermethylation, as used herein, is the presence of methylated alleles in one or more nucleic acids. Nucleic acids from a subject not having a cellular proliferative disorder of gastric tissues contain no detectable methylated alleles when the same nucleic acids are examined.

Gene Marker Names

As used in the present application the following gene symbols are identified as follows. Accession No. is based on the database maintained by NCBI, University of California Santa Cruz (UCSC):

POPDC3, NCBI RefSeq. NM_{—}022361, “Popeye domain-containing protein 3” (SEQ ID NO:12).

CCDC67, NM_{—}181645, “coiled-coil domain containing 67” (SEQ ID NO:13).

LRRC3B, NM_{—}052953, “leucine rich repeat containing 3B” (SEQ ID NO:14).

PRKD1, NM_{—}002742, “protein kinase D1” (SEQ ID NO:15).

CYP1B1, NM_{—}000104, “cytochromoe P450, family 1, subfamily B, polypeptide 1” (SEQ ID NO:16).

LIMS2, NM_{—}017980, “LIM and senescent cell antigen-like domains 2” (SEQ ID NO:17).

DCBLD2, NM080927, “discoidin, CUB and LCCL domain containing 2” (SEQ ID NO:18).

LOC149351, LOC149351, “hypothetical protein LOC149351” (SEQ ID NO:19).

ADCY8, NM_{—}001115, “adenylate cyclase 8” (SEQ ID NO:20).

BACH2, NM_{—}021813, “BTB and CNC homology 1, basic leucine zipper” (SEQ ID NO:21).

ALOX5, NM_{—}000698, “arachidonate 5-lipoxygenase” (SEQ ID NO:22).

TCF4, NM_{—}001083962, “transcription factor 4” (SEQ ID NO:23).

CXXC4, NM_{—}025212, “CXXC finger 4” (SEQ ID NO:24).

CAMK2N2, NM_{—}033259, “CaM-KII inhibitory protein” (SEQ ID NO:25).

EMX1, NM_{—}004097, “empty spiracles homolog 1” (SEQ ID NO:26).

KCNK9, NM_{—}016601, “potassium channel, subfamily K, member 9” (SEQ ID NO:27).

NCAM2, NM_{—}004540, “neural cell adhesion molecule 2 precursor” (SEQ ID NO:28).

AMPD3, NM_{—}000480, “erythrocyte adenosine monophosphate deaminase” (SEQ ID NO:29).

NOG, NM_{—}005450, “noggin precursor” (SEQ ID NO:30).

SP6, NM_{—}199262, “Sp6 transcription factor” (SEQ ID NO:31).

LOC100128675, NR_{—}024561, “hypothetical protein LOC100128675” (SEQ ID NO:32).

CHSY3, NM_{—}175856, “chondroitin sulfate synthase 3”. (SEQ ID NO:33).

Samples

The present application describes early detection of gastric cancer. Gastric cancer specific gene methylation is described. Applicant has shown that gastric cancer specific gene methylation also occurs in tissues that are adjacent to the tumor region. Therefore, in a method for early detection of gastric cancer, any bodily sample, including liquid or solid tissue may be examined for the presence of methylation of the gastric-specific genes. Such samples may include, but not limited to, serum, or plasma.

Primers of the invention are designed to be “substantially” complementary to each strand of the locus to be amplified and include the appropriate G or C nucleotides as discussed above. This means that the primers must be sufficiently complementary to hybridize with their respective strands under conditions that allow the agent for polymerization to perform. Primers of the invention are employed in the amplification process, which is an enzymatic chain reaction that produces exponentially increasing quantities of target locus relative to the number of reaction steps involved (e.g., polymerase chain reaction (PCR)). Typically, one primer is complementary to the negative (−) strand of the locus (antisense primer) and the other is complementary to the positive (+) strand (sense primer). Annealing the primers to denatured nucleic acid followed by extension with an enzyme, such as Taq polymerase and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

Preferably, the method of amplifying is by PCR, as described herein and as is commonly used by those of ordinary skill in the art. However, alternative methods of amplification have been described and can also be employed such as real time PCR or linear amplification using isothermal enzyme. Multiplex amplification reactions may also be used.

Detection of Differential Methylation—Bisulfite Sequencing Method

Another method for detecting a methylated CpG-containing nucleic acid includes contacting a nucleic acid-containing specimen with an agent that modifies unmethylated cytosine, amplifying the CpG-containing nucleic acid in the specimen by means of CpG-specific oligonucleotide primers, wherein the oligonucleotide primers distinguish between modified methylated and non-methylated nucleic acid and detect the methylated nucleic acid. The amplification step is optional and although desirable, is not essential. The method relies on the PCR reaction itself to distinguish between modified (e.g., chemically modified) methylated and unmethylated DNA. Such methods are described in U.S. Pat. No. 5,786,146, the contents of which are incorporated herein in their entirety especially as they relate to the bisulfite sequencing method for detection of methylated nucleic acid.

Substrates

Once the target nucleic acid region is amplified, the nucleic acid can be hybridized to a known gene probe immobilized on a solid support to detect the presence of the nucleic acid sequence.

As used herein, “substrate,” when used in reference to a substance, structure, surface or material, means a composition comprising a nonbiological, synthetic, nonliving, planar, spherical or flat surface that is not heretofore known to comprise a specific binding, hybridization or catalytic recognition site or a plurality of different recognition sites or a number of different recognition sites which exceeds the number of different molecular species comprising the surface, structure or material. The substrate may include, for example and without limitation, semiconductors, synthetic (organic) metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics.

Several types of membranes are known to one of skill in the art for adhesion of nucleic acid sequences. Specific non-limiting examples of these membranes include nitrocellulose or other membranes used for detection of gene expression such as polyvinylchloride, diazotized paper and other commercially available membranes such as GENESCREEN™, ZETAPROBE™ (Biorad), and NYTRAN™. Beads, glass, wafer and metal substrates are included. Methods for attaching nucleic acids to these objects are well known to one of skill in the art. Alternatively, screening can be done in liquid phase.

Hybridization Conditions

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

Label

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

Kit

Invention methods are ideally suited for the preparation of a kit. Therefore, in accordance with another embodiment of the present invention, there is a provided kit useful for the detection of a cellular proliferative disorder in a subject. Invention kits include a carrier means compartmentalized to receive a sample therein, one or more containers comprising a first container containing a reagent which sensitively cleaves unmethylated cytosine, a second container containing primers for amplification of a CpG-containing nucleic acid, and a third container containing a means to detect the presence of cleaved or uncleaved nucleic acid. Primers contemplated for use in accordance with the invention include, but are not limited to, those described in the present application, and any functional combination and fragments thereof. Functional combination or fragment refers to its ability to be used as a primer to detect whether methylation has occurred on the region of the genome sought to be detected.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing methylation sensitive restriction endonuclease. One or more container means can also be included comprising a primer complementary to the locus of interest. In addition, one or more container means can also be included containing an isoschizomer of the methylation sensitive restriction enzyme.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods—Cell Lines and Tissue Samples

The human gastric cancer cell lines used in this study were obtained from the Korean Cell Line Bank and were described previously (23 Park et al., 1990, 24 Park et al., 1997). Fresh gastric tumors paired with normal adjacent tissues were obtained from the Stomach Tissue Bank in Chungnam National University Hospital (CNUH), Daejeon, Korea. Fifteen-paired samples of gastric tumor and normal tissue were used for RLGS analysis. For quantitative gene expression and methylation analysis, 96 paired samples of gastric tumor and normal tissue were used. The samples included 35 TNM stage I, 15 stage II, 33 stage III, and 13 stage IV tumors and were from 30 females and 66 males, 29-82 years of age (average of 58.7 years). Informed consent was obtained from each subject, and their use was approved by the Institutional Review Board of CNUH. All specimens were rapidly frozen in liquid nitrogen and stored at −80° C. until DNA and RNA extraction.

### Example 2

### RLGS Assays

High molecular weight DNA was extracted by a standard protocol and performed RLGS as previously described (19 Hatada et al., 1991). RLGS were run with paired samples of primary tumor and normal tissue. For DNA of cell lines, RLGS were also run in pairs of only cell line DNA and mixed DNA of the cell line with normal tissue to determine the correct position of the spot decreased or lost in RLGS profile of the cell line. Paired RLGS profiles from primary gastric tumors and normal tissues or from cell lines and mixed DNAs and/or normal tissues were overlaid, and the differences between the two profiles were detected by visual inspection and independently validated by two investigators. To exclude a difficulty due to high density or lower resolution of spots and to allow the uniform comparisons of RLGS profiles from different samples, we compared 1,948 spots comprising the central portion of the RLGS profile, which was defined in our previous work (25 Kim et al., 2006).

### Example 3

### Selection of Methylated-NotI-Loci in Gastric Cancer

One of the advantages of using RLGS for methylation analysis is the ability to clone loci of interest using arrayed plasmid libraries. Once a difference in spot intensity was detected between paired normal and tumor sample or normal tissue and cell line, we compared the spots with the previous Master RLGS profile (21 Costello et al., 2000) or our RLGS profile (25 Kim et al., 2006) to get the sequence information.

### Example 4

### Reverse Transcription-PCR

To examine the correspondence between NotI-methylation and gene silencing at the neighboring region of the NotI site, RT-PCR analysis was performed for each selected gene with RNA of gastric cancer cell line. Reverse transcription using 5 μg of DNase-treated RNA was done using Superscript II reverse transcriptase (Invitrogen) in a reaction volume of 20 μL. One μL of the reverse transcription reaction was used for amplification using Platinum Taq DNA polymerase (Invitrogen). Amplification was done as follows: denaturation at 94° C. for 30 s, annealing at a primer specific annealing temperature for 30 s and extension at 72° C. for 45 s. All reactions were performed on a GeneAmp PCR System 9700 (Perkin-Elmer Corp.). Five μL of the PCR product were run on a 0.8% agarose gel and visualized by EtBr staining. GAPDH gene was used as a control for comparison of the amount of reverse transcribed template in each sample.

### Example 5

### Drug Treatment of Cell Lines

Three gastric cancer cell lines, SNU001, SNU601, and SNU668, were treated with 5-aza-2′-deoxycytidine (5-Aza-dC; Sigma) as a demethylating agent and 5-Aza-dC and trichostatin A (TSA; Sigma) as a HDAC inhibitor to examine the restoring of selected genes in those cells. Each cell was plated at a density of 1×10⁵ cells/100-mm dish and cultures for 24 h, followed by 72 h culture with 1 μM 5-Aza-dC. Other cells were also followed by 24 h culture with 250 nM TSA or 72 h 5-Aza-dC-treated cells for another 24 h. RNA was prepared and RT-PCR analysis was then performed using gene-specific primer set as described above. GAPDH gene was also used as a control.

### Example 6

### Quantitative Real-Time RT-PCR in Primary Tumors

The ‘loss of expression level’ (LOE) was quantitatively measured for selected genes or mRNAs in a set of clinical samples by real-time RT-PCR analysis. Total RNAs from 96-paired normal and tumor samples were isolated using Qiagen RNeasy Kit (Qiagen) and first-strand cDNAs were synthesized. The reactions were performed in 96-well based Exicycler apparatus (Bioneer, Korea) using the AccuPower HotStart PCR PreMix (Bioneer, Korea) and SYBR green dye according to manufacturer's instructions. The data was analyzed by using a graphic user interface (GUI)-based operation software supplied by the company. All gene expression levels were expressed as cycle threshold (CT) values, normalized against those of GAPDH. Gene expression level in each tumor was presented relative to that of normal counterpart. Then we arbitrarily labeled each expression level of tumor less than a half of that in paired normal tissue as abnormally LOE.

### Example 7

### Methylation Sensitive-PCR (MS-PCR)

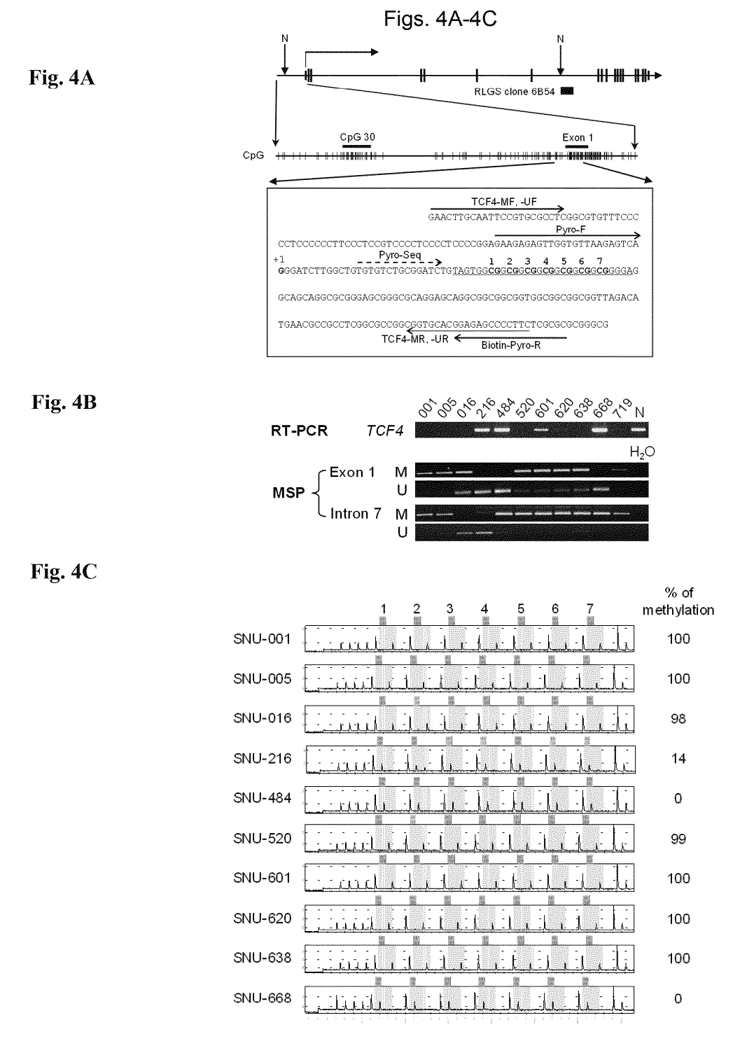

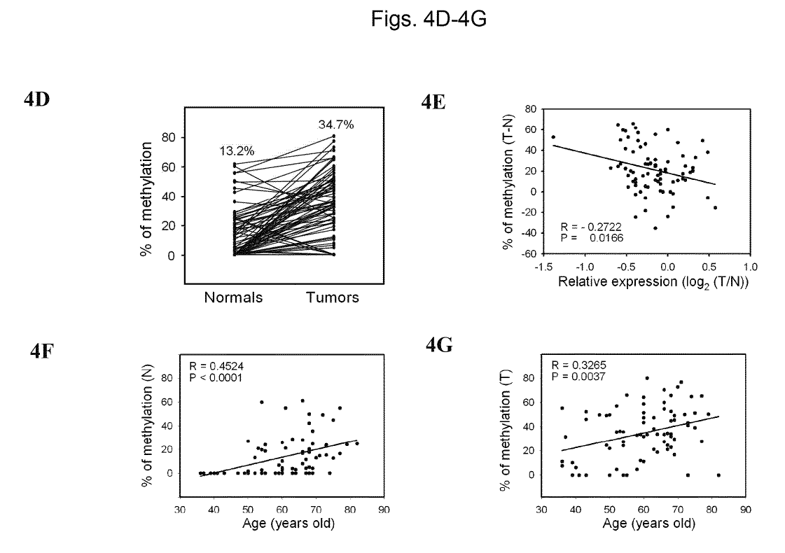

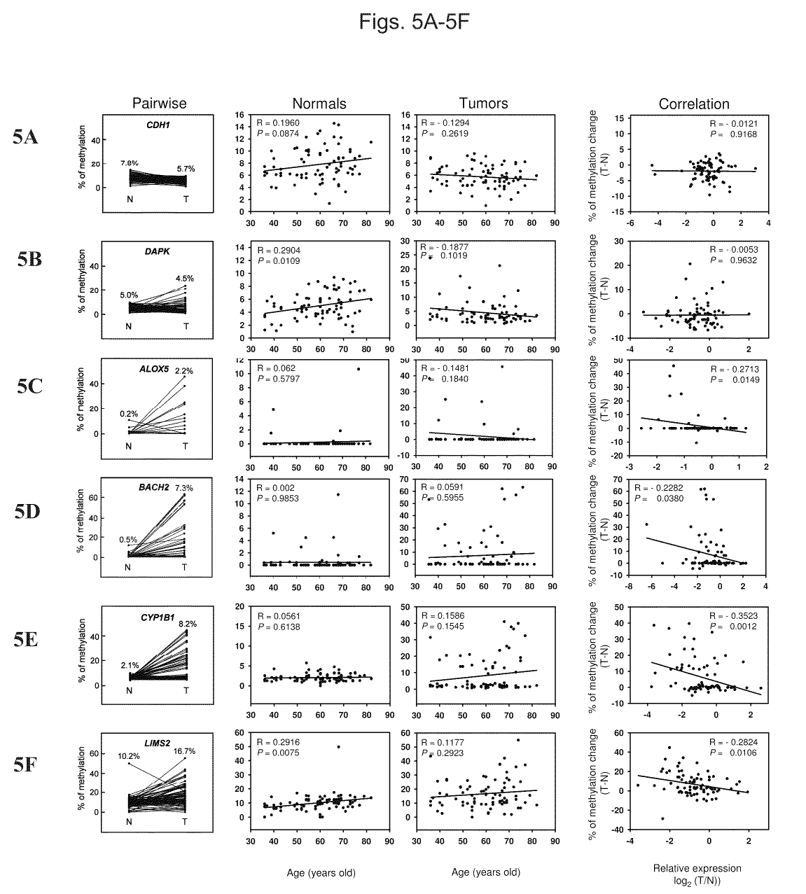

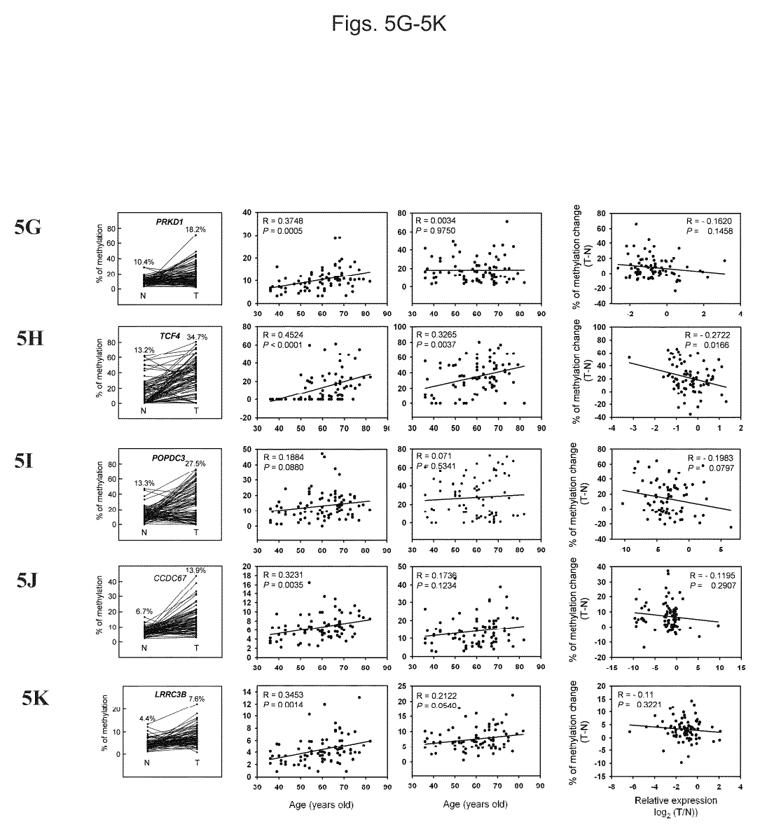

Two genomic regions were chosen to assess the association of methylation with gene silencing of the transcription factor 4 gene (TCF4), a novel epigenetic target: one is from intron 7 a NotI clone lied, and the other one from 5′-upstream region encompassing the exon 1 (FIG. 4A). DNA was modified by sodium bisulfite using the Ez DNA Methylation Kit (ZYMO Research) according the manufacturer's instructions. We designed primers for the methylation specific-PCR (MSP) using MethPrimer program: for methylated sequence of exon 1, TCF4-exon1-MF (forward), 5′-GAATTTGTAATTTCGTGCGTTTC-3′) (SEQ ID NO:1), TCF4-exon1-MR (reverse), 5′-AAAAAAAACTCTCCGTACACCG-3′ (SEQ ID NO:2), and a 258 by product size; for unmethylated sequence of exon 1, TCF4-exon1-UF (forward), 5′-TGAATTTGTAATTTTGTGTGTTTTG-3′ (SEQ ID NO:3), TCF4-exon1-UR (reverse), 5′-AAAAAAAACTCTCCATACACCACC-3′ (SEQ ID NO:4), and a 259 by product size; for methylated sequence of intron 7, TCF4-int7-UF (forward), 5′-TTAATTTTAGAGTGGAGAACGTGC-3′) (SEQ ID NO:5), TCF4-int7-UR (reverse), 5′-AAATAACAATACGACCCGCC-3′ (SEQ ID NO:6), and a 198 by product size; for unmethylated sequence of intron 7, TCF4-int7-UF (forward), 5′-TTTTAGAGTGGAGAATGTGTGT-3′ (SEQ ID NO:7), TCF4-int7-UR (reverse), 5′-AAACAAAATAACAATACAACCCACC-3′ (SEQ ID NO:8), and a 199 by product size. A one tenth to one fifth volume of the bisulfite-modified DNA was amplified in a 20 μL reaction with the primers. All samples were heated to 94° C. for 5 min and then amplified for 35 cycles consisting of 94° C. for 30 s, 59° C. for 30 s, and 72° C. for 60 s. All reactions were then incubated at 72° C. for 7 min and cooled to 4° C. Five μL of each product was then run on a 3% agarose gel and visualized by EtBr staining.

### Example 8

### Quantitative Methylation Analysis by Pyrosequencing

The CpG sites near the transcription start of the TCF4 was chosen for quantitation of methylation using pyrosequencing. A 225-bp fragment containing 25 CpG sites, of which seven were analyzed with one sequencing primer, was amplified in a 50-μL volume using Platinum Taq DNA polymerase (Invitrogen, USA). Mixtures were denatured for 4 min at 95° C. and then thermal-cycled for 30 s at 95° C., 45 s at 50° C., and 20 s at 72° C., repeating the cycle 50 times to ensure complete exhaustion of the primers. A final extension step at 72° C. for 4 min terminated the program. Thermal cycling procedures were carried out in a GeneAmp PCR System 9700 thermal reactor (Perkin-Elmer Corp.). Preparation of ssDNA template was performed from 20-25 μL biotinylated PCR product using streptavidin Sepharose HP beads (Amersham Biosciences, Sweden) following the PSQ 96MA sample preparation guide using multichannel pipets. Sequencing was performed on a PSQ 96MA system with the SNP Reagent Kit (Pyrosequencing AB) according to the manufacturer's instruction. Amplification and sequencing primers were designed with the SNP primer design software (Pyrosequencing AB): for amplification, forward primer, 5′-GAAGAGAGTTGGTGTTAAGAGTTAG-3′ (SEQ ID NO:9) and biotin-labeled reverse primer, 5′-CCACCAAAAAAAACTCTCC-3′ (SEQ ID NO:10); sequencing primer, 5′-TGTGTGTTTGAGGATTTG-3′ (SEQ ID NO:11). The degree of methylation at each CpG site was calculated as allele frequency using the allele quantification functionality of the PSQ 96MA software and the mean value for seven CpG sites was presented as % of methylation for each sample.

### Example 9

### Statistical Analysis

Correlation between the loss of expression and NotI-methylation or quantitative methylation was done using Regression Wizard of SigmaPlot software. Correlations between genes for real-time RT-PCR data in 96-paired samples were done with SAS software (SAS Institute, Cary, N.C.) and plotted as appropriate using SigmaPlot software. The Student's t-test was used to examine clinicopathologic data. Methylation level difference was analyzed by analysis of variance, paired t test, or contingency table as appropriate using SigmaPlot software. The probability of less than 0.05 was considered as significant.

### Example 10

### Results

### Example 10.1.

### Global Methylation of Gastric Cancer Genomes by RLGS

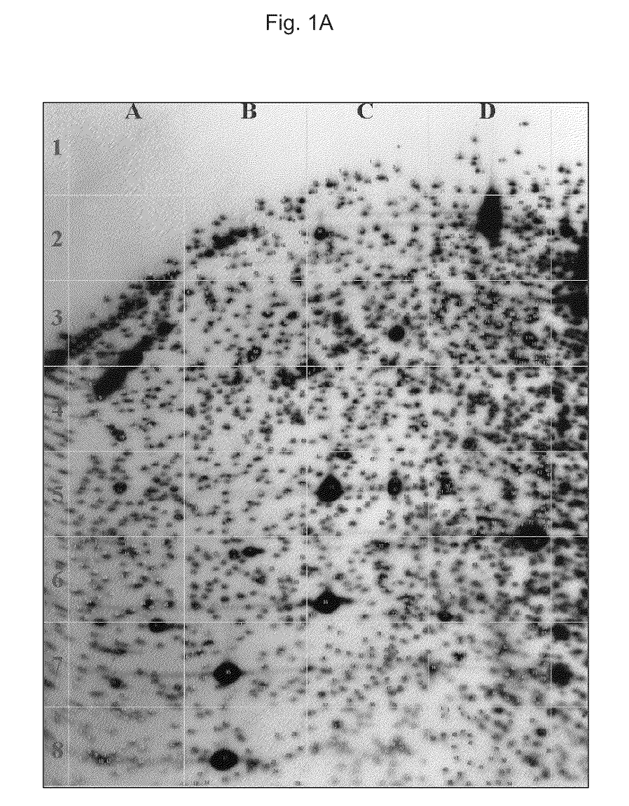

We found that 1.0% to 7.1% (mean 3.3%) of 1,948 spots (FIG. 1A) on the RLGS gel were either absent or had decreased intensity in 15 primary tumors relative to matched normal tissues. Gastric cancer cell lines had from 6.6% to 19.1% (mean 11.9%) missing or decreased intensity spots, showing over three-fold global methylation in cell lines compared to primary tumors. When individual spots were compared, 313 spots were present in normal tissue profiles but absent or decreased in at least one tumor and 929 spots were present in a normal tissue profile but absent or decreased in at least one gastric cancer cell line. In total, 261 spots were found to be absent or decreased concurrently in both of primary tumors and cancer cell lines. FIG. 1B shows the representatives of absent or decreased spots concurrently in both of primary tumors and cancer cell lines.

### Example 10.2.

### Selection of Methylation-Sensitive Genes in Gastric Cancer

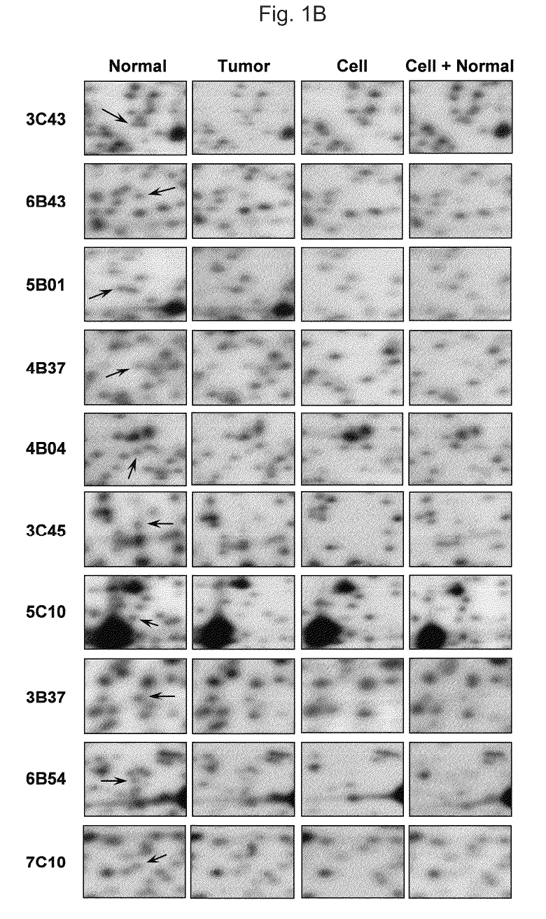

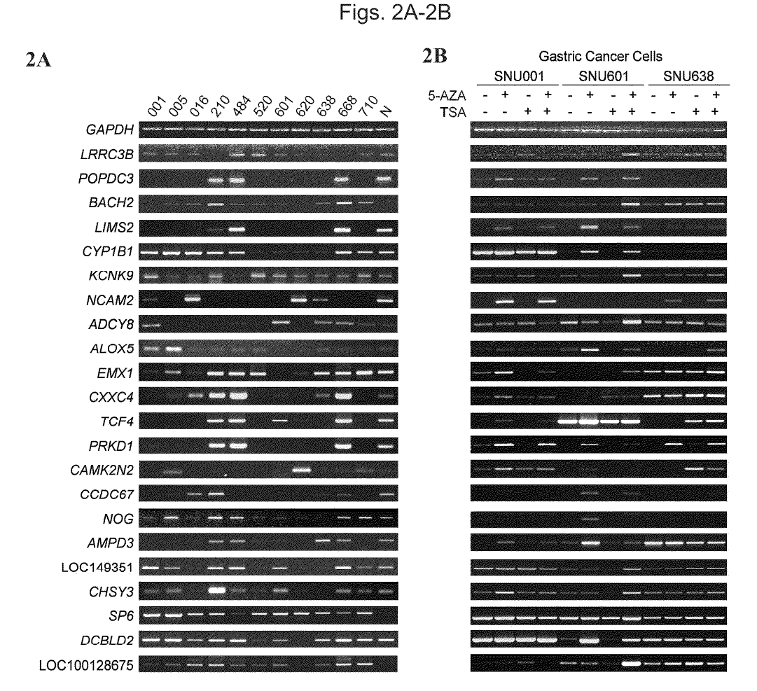

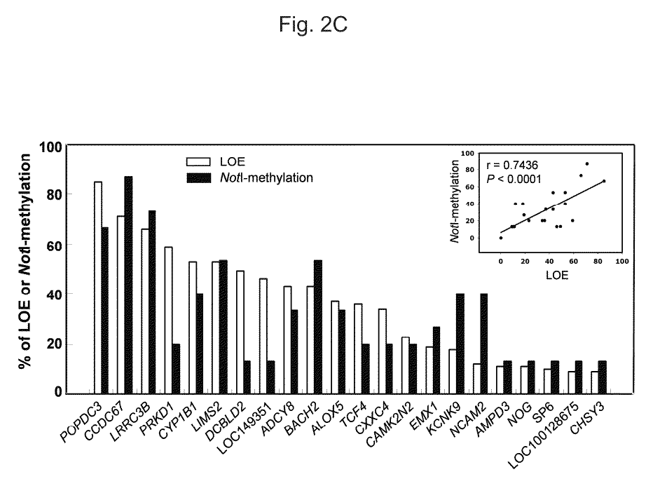

We first selected RLGS spots with changed intensity at least two cancer cells and two tumors coincidentally and labeled according our previous RLGS profile (25 Kim et al., 2006). The spots were compared to those of Master RLGS profile established previously (21 Costello et al., 2000) and also labeled as Master RLGS spot number only when a spot position was correctly matched with each other. In total, we identified 40 spots for which sequence information was available in previous literatures (Table 1): of the 40 spots, 29 came from cloned NotI-linked sequences in our previous study (25 Kim et al., 2006) and the remaining 11 from previous RLGS works (21 Costello et al., 2000; 22 Rush et al., 2004; 26 Smiraglia et al., 2001; 27 Rush et al., 2001; 28 Dai et al., 2001). We next examined the variability of gene expression across 11 gastric cancer cell lines using RT-PCR analysis and the coincidence of the gene expression with the methylation status from RLGS data. Only 29 genes or mRNAs showed variable expression level across gastric cancer cells (FIG. 2A). On the other hand, remaining three genes showed full expression (no variability) across cell lines and eight had no PCR product. In summary, 22 of 29 genes showing variability coincided with RLGS data in gastric cancer cell lines.

### Example 10.3

### Reactivation Analysis by Drug Treatment

For the selected 22 genes, reactivation analysis was examined in three gastric cancer cell lines by 5-Aza-dC and/or TSA treatment. We observed the genes to be fully or partially restored in various modes: for example, POPDC3, NACM2, PRKD1 and AMPD3 were restored in two or three corresponding inactivated cells only by 5-Aza-dC; CYP1B1, CXXC4, TCF4, CAM-KIIN and CCDC67 in one cell by 5-Aza-dC and in other cell by TSA; the remaining genes such as LRRC3B in several cells by 5-Aza-dC and/or TSA (FIG. 2A). The result suggests that the genes selected in this study could be epigenetic targets altered in gastric cancer cells by gene-specific or cell-specific modes.

### Example 10.4.

### Combined Expression of Novel Epigenetic Targets by Quantitative Real-Time PCR

For the selected 22 genes, we analyzed their relative expression levels in 96-paired normal and tumor samples using quantitative real-time PCR and observed the LOE within the range of 10˜85% in primary tumors (see the last column in Table 1). In addition, the LOE of DAPK and CDH1, which are well-known as tumor suppressor genes in gastric cancer, were also examined in the same 96-paired samples for comparison with those of selected genes. The LOE level was analyzed to be 51% and 39% in DAPK and CDH1, respectively. As the LOE level of each gene were compared with the degrees of related spot decrease from RLGS data, we found a high correlation between the two values (r=0.7436, P<0.0001) (Table 1 and FIG. 2B), though difference was observed in PRKD1, DCBLD2, LOC149351, KCNK9 and NCAM2.

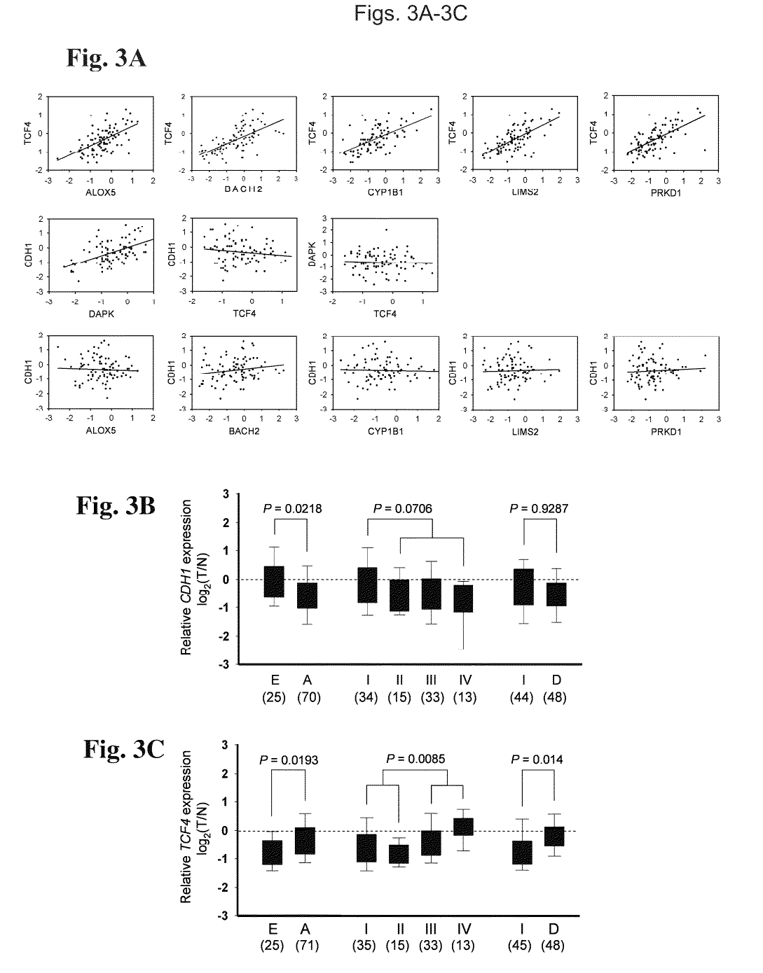

For 15 genes showing over 30% of LOE including DAPK and CDH1, the pairwise correlation of gene expression between these genes was examined in 96 paired samples. We found a strong correlation between DAPK and CDH1 (Table 2), but these genes showed no correlation against any epigenetic targets selected in this study. Instead, a significant correlation was observed among the 13 selected genes, indicating that novel epigenetic targets were expressed among the clinical samples in similar pattern to each other, but independent of DAPK and CDH1. FIG. 3A shows a strong correlation for PRKD1 (r=0.62, P<0.0001), CYP1B1 (r=0.60, P<0.0001), LIMS2 (r=0.70, P<0.0001), ALOX5 (r=0.67, P<0.0001), and BACH2 (r=0.67, P<0.0001) against TCF4 and another correlation between CDH1 and DAPK (r=0.63, P<0.0001). But CDH1 or DAPK showed no correlation with PRKD1, CYP1B1, LIMS2, ALOX5, BACH2, and TCF4.

### Example 10.5.

### Comparison of TCF4 and CDH1 Status in Gastric Carcinogenesis

We selected TCF4 and CDH1 to elucidate why the expression pattern are different between two groups during gastric carcinogenesis. Quantitative real-time RT-PCR showed that the LOE level of CDH1 was high in advanced gastric cancer type (30 of 70, 43%) compared with early gastric cancer type (6 of 19, 24%) or higher in later TNM stage (28 of 61, 50% in stage II˜IV) than early TNM stage (10 of 34, 29% in stage I) (FIG. 3B). No significant difference was found between intestinal type (17 of 44, 38%) and diffuse type (18 of 48, 37%). On the contrary, the LOE level of TCF4 was significantly higher in early gastric type (15 of 25, 60%) compared with advanced gastric tumors (20 of 71, 28%; P=0.0045) or significantly higher in earlier TNM stage (25 of 50, 50% in stage I and II) than in later stage (10 of 46, 22% in stage III and IV; P=0.0041). The abnormal reduction of TCF4 was also significantly high in intestinal type (27 of 45, 60%) rather than diffuse type (8 of 48, 17%; P=0.0001) (FIG. 3C). No significant difference was found between different genders or age groups (data not shown). Thus, this result shows that two genes were dysregulated with a different stage-dependent mode in primary gastric tumor.

### Example 10.6.

### Association of TCF4 Gene Silencing With Hypermethylation on TCF4 Exon 1

Of the selected genes, we chose the TCF4 gene to certify a correlation gene silencing with epigenetic modification. We first performed MSP analysis based on DNA sequence of cloned NotI-linked sequence (spot #6B54) in the 7th intron of the TCF4 gene (FIG. 4A) and found that 7 of 11 cell lines had a positive correlation between methylation and TCF4 silencing. At the same time, we found more close correlation in 10 cell lines except SNU601, when MSP analysis was performed on exon 1 of TCF4 (FIG. 4A, C). Then we measured quantitatively methylation status on seven CpG sites of exon 1 using pyrosequencing analysis (FIG. 4A). Six cell lines having no transcript of TCF4 (SNU001, SNU005, SNU016, SNU520, SNU620, and SNU638) showed heavy methylation of 98 to 100% on the sites (FIG. 4C). On the other hand, the methylation status of three cell lines (SNU216, SNU484, and SNU668) with strong TCF4 expression ranged of 0 to 14%. This result indicates a strong association of hypermethylation on TCF4 exon 1 with gene silencing on most of gastric cancer cells, except a SNU601 cell which expressed TCF4 but the sites were heavily methylated.

### Example 10.7.

### Hypermethylation of TCF4 Exon 1 in Primary Gastric Tumors

We next measured quantitatively methylation status on the above seven CpG sites using pyrosequencing with 85 paired normal and tumor DNAs, which matched to samples used for quantitative expression analysis. Seven patient samples failed to produce good Pyrogram in normal, tumor or both DNAs and the samples were excluded from further analysis. The result showed mean methylation of 10.9%, 13.8%, 13.5%, 15.1%, 12.4%, 13.3%, and 13.3% in each CpG site for 77 normal DNA samples. Thus, the degree of methylation can be calculated as 13.2% in average from seven CpG sites (FIG. 4D). On the other hand, 77 tumor DNAs showed 34.9%, 34.1%, 34.5%, 34.2%, 34.3%, 35.4%, and 35.8% on each CpG site and thus 34.7% average methylation, showing a significant difference compared to normal tissues (t-test, P<0.0001) (FIG. 4D). To elucidate whether the methylation of TCF4 exon 1 is associated with abnormal expression for the clinical samples, we examined a correlation between methylation change and relative expression level. For this analysis, we arbitrarily defined the degree of methylation in tumor DNA minus that in normal DNA as methylation change in each paired DNA. A significant negative correlation was found between methylation change and relative expression level (R=−0.2722, P=0.0166), indicating that the hypermethylation of TCF4 exon 1 was associated with LOE (FIG. 4E).

### Example 10.8.

### Age-Related Methylation of TCF4 Exon 1

When the degree of methylation was compared within each clinicopathologic category, we observed no significant difference in mean percentage of methylation between early (EGC) and advanced gastric (AGC) types: for normal tissues, 12.6% in EGC (N=18) and 13.3% in AGC (N=59); for tumor tissues, 35.8% in EGC and 34.4% in AGC (see the left figure in FIG. 4F). No significant differences in mean percentage of methylation were found between groups of other parameters, such as tumor stage or Lauren's classification, though tumors of intestinal type (36.9%, N=40) showed higher methylation than that of diffuse type (32.4%, N=37) (see the center figure in FIG. 4F). For both normal and tumor tissues, however, we found a gradual methylation along with age: for age group 1 (≦50 years), the mean methylation of TCF4 exon 1 was 1.7% and 24.5% in normal and tumor DNAs (N=17); for age group 2 (51˜60 years), 9.5% and 30.9% (N=22); for age group 3 (61˜70 years), 18.2% and 41.0% (N=26); for age group 4 (>70 year), 25.3% and 42.5% (N=12), respectively (see the right figure in FIG. 4F). FIG. 4G shows that the methylation on TCF4 exon 1 dramatically increases with the patient's age in normal tissues (R=0.4524, P<0.0001) as well as in tumor tissues (R=0.3265, P=0.0037). This result suggests that TCF4 exon 1 is significantly methylated with age-related as well as cancer-specific mode. In addition, it is worthy to note that the patients (N=14) before 50 years had zero % of methylation on TCF4 exon 1 in normal tissues but that the normal tissues of the patients (N=12) over 70 years had mean methylation status (25.3%) close to that (24.5%) from tumor tissues below 50 years (N=17).

### REFERENCES

1. Zheng L, Wang L, Ajani J, Xie K. Molecular basis of gastric cancer development and progression. Gastric Cancer 2004;7:61-77.

2. Parkin D M, Bray F, Ferlay J, Pisani P. Global cancer statistics, 2002. CA Cancer J Clin 2005;55:74-108.

3. Zardo G, Tiirikainen M I, Hong C, et al. Integrated genomic and epigenomic analyses pinpoint biallelic gene inactivation in tumors. Nat Genet 2002;32:453-8.

4. Jones P A, Baylin S B. The fundamental role of epigenetic events in cancer. Nat Rev Genet 2002;3:415-28.

5. Laird P W. The power and the promise of DNA methylation markers. Nat Rev Cancer 2003;3: 253-66.

6. Momparler R L, Bouffard D Y, Momparler L F, Dionne J, Belanger K, Ayoub J. Pilot phase I-II study on 5-aza-2′-deoxycytidine (Decitabine) in patients with metastatic lung cancer. Anticancer Drugs 1997;8:358-68.

7. Pohlmann P, DiLeone L P, Cancella A I, et al. Phase II trial of cisplatin plus decitabine, a new DNA hypomethylating agent, in patients with advanced squamous cell carcinoma of the cervix. Am J Clin Oncol 2002;25:496-501.

8. Brown R, Strathdee G. Epigenomics and epigenetic therapy of cancer. Trends Mol Med 2002; 8:S43-48.

9. Lauren P. The two histological main types of gastric carcinoma: diffuse and so-called intestinal-type carcinoma. An attempt at a histo-clinical classification. Acta Pathol Microbiol Scand 1965;64:31-49.

10. Tamura G, Maesawa C, Suzuki Y, et al. Mutations of the APC gene occur during early stages of gastric adenoma development. Cancer Res 1994;54:1149-51.

11. Clement G, Bosman F T, Fontolliet C, Benhattar J. Monoallelic methylation of the APC promoter is altered in normal gastric mucosa associated with neoplastic lesions. Cancer Res 2004;64:6867-73.

12. Becker K F, Atkinson M J, Reich U, et al. E-cadherin gene mutations provide clues to diffuse type gastric carcinomas. Cancer Res 1994;54:3845-52.

13. Oue N, Oshimo Y, Nakayama H, et al. DNA methylation of multiple genes in gastric carcinoma: association with histological type and CpG island methylator phenotype. Cancer Sci 2003;94:901-5.

14. Iida S, Akiyama Y, Nakajima T, et al. Alterations and hypermethylation of the p14(ARF) gene in gastric cancer. Int J Cancer 2000;87:654-8.

15. Li Q L, Ito K, Sakakura C, et al. Causal relationship between the loss of RUNX3 expression and gastric cancer. Cell 2002;109:113-24.

16. Toyota M, Ahuja N, Ohe-Toyota M, Herman J G, Baylin S B, Issa J P. CpG island methylator phenotype in colorectal cancer. Proc Natl Acad Sci USA 1999;96:8681-6.

17. Toyota M, Ahuja N, Suzuki H, et al. Aberrant methylation in gastric cancer associated with the CpG island methylator phenotype. Cancer Res 1999;59:5438-42.

18. Lee J H, Park S J, Abraham S C, et al. Frequent CpG island methylation in precursor lesions and early gastric adenocarcinomas. Oncogene 2004;23:4646-54.

19. Hatada I, Hayashizaki Y, Hirotsune S, Komatsubara H, Mukai T. A genomic scanning method for higher organisms using restriction sites as landmark. Proc Natl Acad Sci USA 1991;88:9523-27.

20. Nagai H, Ponglikitmongkol M, Mita E, et al. Aberration of genomic DNA in association with human hepatocellular carcinomas detected by 2-dimensional gel analysis. Cancer Res 1994;54: 1545-50.

21. Costello J F, Fruhwald M C, Smiraglia D J, et al. Aberrant CpG-island methylation has non-random and tumor-type-specific patterns. Nat Genet 2000;24:132-8.

22. Rush L J, Raval A, Funchain P, et al. Epigenetic profiling in chronic lymphocytic leukemia reveals novel methylation targets. Cancer Res 2004;64:2424-33.

23. Park J G, Frucht H, LaRocca R V, et al. Characteristics of cell lines established from human gastric carcinoma. Cancer Res 1990;50:2773-80.

24. Park J G, Yang H K, Kim W H, et al. Establishment and characterization of human gastric carcinoma cell lines. Int J Cancer 1997;70:443-9.

25. Kim J H, Lee K T, Kim H C et al. Cloning of NotI-linked DNA detected by restriction landmark genomic scanning of human genome. Genomics & Informatics 2006; 4:18-27.

26. Smiraglia D J, Rush L J, Fruhwald M C, Dai Z, Held W A, Costello J F, Lang J C, Eng C, Li B, Wright F A, Caligiuri M A, Plass C. Excessive CpG island hypermethylation in cancer cell lines versus primary human malignancies. Hum Mol Genet. 2001:10:1413-9.

27. Rush L J, Dai Z, Smiraglia D J, Gao X, Wright F A, Fruhwald M, Costello J F, Held W A, Yu L, Krahe R, Kolitz J E, Bloomfield C D, Caligiuri M A, Plass C. Novel methylation targets in de novo acute myeloid leukemia with prevalence of chromosome 11 loci. Blood 2001;97:3226-33.

28. Dai Z, Lakshmanan R R, Zhu W G, Smiraglia D J, Rush L J, Fruhwald M C, Brena R M, Li B, Wright F A, Ross P, Otterson G A, Plass C. Global methylation profiling of lung cancer identifies novel methylated genes. Neoplasia 2001;3:314-23.

29. Nakamura M, Konishi N, Tsunoda S, Hiasa Y, Tsuzuki T, Aoki H, Kobitsu K, Nagai H, Sakaki T. Analyses of human gliomas by restriction landmark genomic scanning. J Neurooncol. 1997;35:113-20.

30. Cho M, Konishi N, Yamamoto K, Inui T, Kitahori Y, Nakagawa Y, Uemura H, Hirao Y, Hiasa Y. Genomic aberrations in renal cell carcinomas detected by restriction landmark genomic scanning. Eur. J. Cancer 34, 2112-2118 (1998).

31. Grady W M. Epigenetic events in the colorectum and in colon cancer. Biochem Soc Trans. 2005;33:684-8.

32. Paz M F, Fraga M F, Avila S, Guo M, Pollan M, Herman J G, Esteller M. A systematic profile of DNA methylation in human cancer cell lines. Cancer Res. 2003;63:1114-21.

33. Esteller M, Corn P G, Baylin S B, Herman J G. A gene hypermethylation profile of human cancer. Cancer Res. 2001;61:3225-9.

34. Cai J, Ikeguchi M, Tsujitani S, Maeta M, Liu J, Kaibara N. Significant correlation between micrometastasis in the lymph nodes and reduced expression of E-cadherin in early gastric cancer. Gastric Cancer. 2001;4(2):66-74.

35. Henthorn, P.; Kiledjian, M.; Kadesch, T. Two distinct transcription factors that bind the immunoglobulin enhancer mu-E5/kappa-E2 motif. Science 247: 467-470, 1990.

36. Corneliussen, B.; Thornell, A.; Hallberg, B.; Grundstrom, T. Helix-loop-helix transcriptional activators bind to a sequence in glucocorticoid response elements of retrovirus enhancers. J. Virol. 65: 6084-6093, 1991.

37. Kolligs F T, Nieman M T, Winer I, Hu G, Van Mater D, Feng Y, Smith I M, Wu R, Zhai Y, Cho K R, Fearon E R. ITF-2, a downstream target of the Wnt/TCF pathway, is activated in human cancers with beta-catenin defects and promotes neoplastic transformation. Cancer Cell 2002;1:145-55.

38. Slaughter D P, Southwick H W, Smejkal W. Field cancerization in oral stratified squamous epithelium; clinical implications of multicentric origin. Cancer 1953; 6: 963-8.

39. Braakhuis B J, Tabor M P, Kummer J A, Leemans C R, Brakenhoff R H. A genetic explanation of Slaughter's concept of field cancerization: evidence and clinical implications. Cancer Res 2003; 63: 1727-30.

40. J. P. Issa, Y. L. Ottaviano, P. Celano, S. R. Hamilton, N. E. Davidson, S. B. Baylin, Methylation of the oestrogen receptor CpG island links ageing and neoplasia in human colon, Nat. Genet. 7 (1994) 536-540.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of diagnosing gastric cancer or a stage in the progression of the cancer in a subject comprising assaying for loss of expression of a marker gene, which is selected from the group consisting of: POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, and CHSY3, or a combination thereof.

2. The method of claim 1, wherein the loss of expression is caused by hypermethylation of the marker gene.

3. The method of claim 2, wherein the hypermethylation occurs in a regulatory region or an amino acid encoding region.

4. The method of claim 1, wherein the stage is early TNM (Tumor, Node, Metastasis) stage.

5. The method of claim 4, wherein the TNM stage is stage I.

6. The method of claim 4, wherein the marker gene is TCF4, PRKD1, CYP1B1, LIMS2, ALOX5, or BACH2, or a combination thereof.

7. The method of claim 6, wherein the marker gene is TCF4.

8. The method of claim 7, wherein methylation of TCF4 occurs in exon I.

9. The method of claim 1, wherein the gastric cancer is intestinal type.

10. The method of claim 9, wherein the marker gene is TCF4, PRKD1, CYP1B1, LIMS2, ALOX5, or BACH2, or a combination thereof.

11. The method of claim 10, wherein the marker gene is TCF4.

12. The method of claim 11, wherein methylation of TCF4 occurs in exon I.

13. A method of diagnosing likelihood of developing gastric cancer comprising assaying for methylation of a gastric cancer specific marker gene in normal appearing bodily sample.

14. The method of claim 13, wherein the bodily sample is solid tissue, or body fluid.

15. The method of claim 13, wherein the marker gene is TCF4, PRKD1, CYP1B1, LIMS2, ALOX5, or BACH2, or a combination thereof.

16. A kit comprising
(i) a carrier means compartmentalized to receive a sample therein, and
(ii) one or more containers comprising a first container containing a reagent which sensitively cleaves unmethylated cytosine, a second container containing primers for amplification of a CpG-containing nucleic acid, and a third container containing a means to detect the presence of cleaved or uncleaved nucleic acid.

17. The kit of claim 16, wherein the nucleic acid is a marker gene for detection of gastric cancer.

18. The kit of claim 17, wherein the marker gene is POPDC3, CCDC67, LRRC3B, PRKD1, CYP1B1, LIMS2, DCBLD2, LOC149351, ADCY8, BACH2, ALOX5, TCF4, CXXC4, CAMK2N2, EMX1, KCNK9, NCAM2, AMPD3, NOG, SP6, LOC100128675, or CHSY3, or a combination thereof.

19. The kit of claim 18, wherein the nucleic acid is a marker gene for detection of early gastric cancer.

20. The kit of claim 19, wherein the marker gene is TCF4, PRKD1, CYP1B1, LIMS2, ALOX5, or BACH2, or a combination thereof.
